SEQUENCE LISTING (16 sequences)

SEQ ID NO: 1 - RNA, 57 nt, Artificial
  Synthetic oligonucleotide shRNA
ggagaucacacugaccuggcauuuguguagugccaggucagugugaucuccuuuuuu

SEQ ID NO: 2 - RNA, 57 nt, Artificial
  Synthetic oligonucleotide shRNA
ggagaucacguguaccuggcauuuguguagugccagguacacgugaucuccuuuuuu

SEQ ID NO: 3 - RNA, 57 nt, Artificial
  Synthetic oligonucleotide shRNA
caccugccaugugcagcaugauuuguguagucaugcugcacauggcagguguuuuuu

SEQ ID NO: 4 - DNA, 28 bp, Artificial
  primer
cgtgcacagggtgtcacgttgcaagacc

SEQ ID NO: 5 - DNA, 28 bp, Artificial
  primer
cctcgtattgggaatccccgaacatcgc

SEQ ID NO: 6 - DNA, 20 bp, Artificial
  probe
cgatcttagccagacgagcg

SEQ ID NO: 7 - DNA, 20 bp, Artificial
  probe
ctggcaaactgtgatggacg

SEQ ID NO: 8 - DNA, 20 bp, Artificial
  probe
cctcgtgcacgcggatttcg

SEQ ID NO: 9 - protein, 9 aa, Artificial
  HLA A2-restricted peptide
GILGFVFTL

SEQ ID NO: 10 - protein, 11 aa, Artificial
  HLA A2-restricted peptide
FVFTLTVPSER

SEQ ID NO: 11 - DNA, 29 bp, Artificial
  primer
tcttcctctacacagcaagctcaccgtgg

SEQ ID NO: 12 - DNA, 24 bp, Artificial
  primer
gagggttcttccttctcggctttc

SEQ ID NO: 13 - DNA, 25 bp, Artificial
  probe
ttcactctgaagaagatgcctagcc

SEQ ID NO: 14 - DNA, 20 bp, Artificial
  primer
acacaactgtgttcactagc

SEQ ID NO: 15 - DNA, 20 bp, Artificial
  primer
gtctccttaaacctgtcttg

SEQ ID NO: 16 - DNA, 23 bp, Artificial
  probe
acctgactcctgaggagaagtct

(19) United States
(12) Patent Application Publication
(10) Pub. No.: US 2007/0036773 A1
(43) Pub. Date: Feb. 15, 2007

(54) Generation and application of universal T cells for B-ALL

(75) Inventors: Laurence Cooper, Houston, TX (US); John J. Rossi, Alta Loma, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 11/500,490

(22) Filed: Aug. 8, 2006

Related U.S. Application Data
(60) Provisional application No. 60/706,423, filed on Aug. 9, 2005.

(51) Int. Cl. A61K 48/00; C12N 5/08

(52) U.S. Cl. 424/93.21; 435/372

(57) ABSTRACT

The present invention is directed to universal T cells and their use in treating diseases and other physiological conditions. More specifically, the present invention is directed to universal T cells and their use in treating treating B-lineage acute lymphoblastic leukemia (B-ALL) in particular and malignancy in general. The universal T cells contain (i) nucleic acid encoding a chimeric antigen receptor (CAR) to redirect their antigen specificity and effector function and (ii) nucleic acids encoding shRNA and/or siRNA molecules to down-regulate cell-surface expression of T cell classical HLA class I and/or II genes to avoid recognition by recipient T cells. The universal T cells may also contain a nucleic acid encoding a non-classical HLA gene, such as an HLA E gene to enforce expression of HLA E genes and/or an HLA G gene to enforce expression of HLA G genes, to avoid recognition by recipient NK cells. The universal T cells may further contain a nucleic acid encoding a selection-suicide gene.

### CROSS-REFERENCE TO RELATED APPLICATION

The present application is related to the claims and priority under 35 U.S.C. § 119 (e) to U.S. provisional patent application Ser. No. 60/706,423 filed 9 Aug. 2005, incorporated herein by reference.

### STATEMENT REGARDING FEDERALLY SPONSORED RESEARCH

This application was made with Government support under Grant No. NCI PO1 CA30206 funded by the National Institutes of Health, Bethesda, Md. The federal government may have certain rights in this invention.

### BACKGROUND OF THE INVENTION

The present invention is directed to universal T cells and their use in treating diseases and other physiological conditions. More specifically, the present invention is directed to universal T cells and their use in treating B-lineage acute lymphoblastic leukemia (B-ALL) in particular and malignancy in general. The universal T cells contain (i) nucleic acid encoding a chimeric antigen receptor (CAR) to redirect their antigen specificity and effector function and (ii) nucleic acids encoding shRNA and/or siRNA molecules to down-regulate cell-surface expression of T cell classical HLA class I and/or II genes to avoid recognition by recipient T cells. The universal T cells may also contain a nucleic acid encoding a non-classical HLA gene, such as an HLA E gene to enforce expression of HLA E genes and/or an HLA G gene to enforce expression of HLA G genes, to avoid recognition by recipient NK cells. The universal T cells may further contain a nucleic acid encoding a selection-suicide protein.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

As supportive care measures have improved, relapse has emerged as the major impediment to improving the outcome of patients with acute lymphoblastic leukemia (ALL). The inability of maximally intensive regimens to eradicate minimal residual disease (MRD) is the mechanism of treatment failure after chemotherapy, radiation therapy and hematopoietic stem-cell transplantation (HSCT). Relapsed ALL is difficult to cure as patients' response to salvage therapy is typically of shorter duration after each relapse, and the prognosis is generally death as a result of disease-related causes. Patients with low complete response rates or high incidence of early relapse are at high risk since they fare very poorly and have a short median survival. It is this group of patients that require treatment with innovative approaches.

The majority of ALL are of B-cell origin, accounting for 50% of ALL's in adults and 70% in children (Foon et al., 1986; Pui, 1995; Pui et al., 2004). Conventional therapeutic modalities for ALL are curable in only 20-35% of adults, compared with 80% to 90% in children (Berger et al., 2000; York et al., 1994). Relapsed ALL remains a significant challenge for pediatric oncologists, however, as this disease is a common malignant diagnosis made in children. The prognosis for patients who suffer a relapse, is poor with salvage chemotherapy alone (Tanchot et al., 1997; Shen and Konig, 2001; Mackall et al., 1996) and the survival of patients in second relapse is poor. Allogeneic HSCT from a related or unrelated donor can salvage a significant proportion of high-risk patients (Freitas et al., 1996; Correia-Neves et al., 2001; Berenson et al, 1975; Eberlein et al., 1982; Maine and Mule, 2002). However, the 5-year DFS remains only approximately 50%. With the exception of second transplants for selected children, there is no effective salvage therapy for adults with ALL when it recurs following HSCT (Maine and Mule, 2002).

Adoptive immunotherapy can be used to overcome tolerogenic mechanisms by enabling the selection and activation of highly reactive T cell subpopulations and by manipulation of the host environment into which the T cells are introduced. For example, adoptive immunotherapy can reduce the complications of viral infection after allogeneic HSCT. Clinical trials have demonstrated that adoptively transferred ex vivo-expanded donor-derived T cell lines specific for Epstein-Barr virus (EBV) can protect patients at high risk for development of EBV lymphoproliferative disease as well as mediate the eradication of clinically evident EBV-transformed B cells (Heslop and Rooney, 1997). In addition, the safety of adoptively transferring CD8⁺ CMV-specific T cell clones has been established in allogeneic bone marrow transplant recipients who received donor-derived HLA-matched CMV-specific T cells in an effort to reconstitute deficient CMV immunity following BMT (Walter et al., 1995). The recoverable CMV-specific cytolytic T lymphocyte (CTL) activity increased after each successive T cell infusion, and persisted at least 3 months after the last infusion, although long-term persistence of CD8⁺ T cell clones was not observed without a concurrent CD4⁺ helper response (Heslop and Rooney, 1997; Walter et al., 1995).

Non-transformed B-cells and malignant B-cells express an array of cell-surface molecules that define their lineage commitment and stage of maturation. CD19 is expressed on all human B-cells beginning from the initial commitment of stem cells to the B lineage and persisting until terminal differentiation into plasma cells. CD19 is a type I transmembrane protein that associates with the complement 2 (CD21), TAPA-1, and Leu13 antigens forming a B-cell signal transduction complex. This complex participates in the regulation of B-cell proliferation (Stamenkovic and Seed, 1988). CD19 is expressed on the majority of adult and pediatric ALLs. In vitro progenitor assays have indicated that progenitor cells of ALL express CD19 (Stamenkovic and Seed, 1988). Although CD19 does not shed from the cell surface, it does internalize (Freitas et al., 1996; Correia-Neves et al., 2001). Accordingly, targeting CD19 with monoclonal antibodies conjugated to liposomes (Lopes de Menezes et al., 2000; Sapra et al., 2004), immunotoxin (Dinndorf et al., 2001; Longo et al., 2000; Roy et al., 1995; Szatrowski et al., 2003; Tsimberidou et al., 2003), and radionuclides (Ma et al., 2002; Mitchell et al., 2003) is currently being investigated as a strategy to specifically deliver cytotoxic agents to the intracellular compartment of malignant B-cells. Anti-CD19 antibody conjugated to blocked ricin and poke-weed antiviral protein (PAP) dramatically increase specificity and potency of leukemia cell killing both in ex vivo bone marrow purging procedures and when administered to NOD/scid animals inoculated with CD19⁺ leukemia cells (Longo et al., 2000). CD19 has also been targeted by CD3xCD19 bi-specific antibody-conjugates to target polyclonal T cells to malignant cells (Roy et al., 1995; Szatrowski et al., 2003; Tsimberidou et al., 2003). Recently, a chimeric CD19 antibody has been used to induce antibody-dependent cellular cytotoxicity of NK cells recovered after TCD allogeneic HCT (Ma et al., 2002).

Studies evaluating the biology of T cell antigen receptor signal transduction revealed that cross-linking chimeric molecules consisting of the extracellular domain of CD8, fused to the intracellular domain of the CD3 complex zeta chain, resulted in activation of T cell hybridomas mimicking that of the endogenous TCR complex (Irving and Weiss, 1991; Chan et al., 1991). Concurrently, engineered immunoglobulin molecules consisting of single-chain variable regions joined by flexible amino acid linkers were shown to assume conformations capable of antigen binding (Bird et al., 1988; Eshhar et al., 1993; Hekele et al., 1996). Chimeric antigen receptors evolved from the fusing of extracellular single-chain antibodies to the intracellular domain of CD3-ζ or FcγRIII chain. These chimeric antigen receptors (CARs, scFvFc:ζ) are distinguished by their ability to both bind antigen and transduce activation signals via immunoreceptor tyrosine-based activation motifs (ITAM's) present in their cytoplasmic tails. The genetic modification of T cells to synthesize a scFvFc:ζ for re-directed antigen specificity is one strategy to generate effector cells for adoptive therapy that does not rely on pre-existing anti-tumor T cell immunity and overcomes many of the limitations of the bispecific antibody approach. These receptors are “universal” in that they bind antigen in an HLA-independent fashion, thus, one receptor construct can be used to treat a population of patients with antigen positive tumors. A growing number of constructs for targeting human tumors have been described in the literature, including receptors with specificity for Her2/Neu, TAG-72, CEA, ErbB-2, CD44v6, as well as the B-cell targets CD20 and CD19 (Cooper et al., 2003; Brocker and Karjalainen, 1998; Eshhar, 1997; Jensen et al., 1998; U.S. Pat. No. 6,410,319; U.S. published patent application No. 2004/0126363 A1). These epitopes all share the common characteristic of being cell-surface moieties accessible to scFv binding by the chimeric T cell receptor (TCR). Animal models have demonstrated the capacity of adoptively transferred scFvFc:ζ-expressing T cells to eradicate established tumors in vivo (Hekele et al., 1996; Altenschmidt et al., 1997; Hu et al., 2002; McGuinness et. al., 1999). scFvFc:ζ⁺ CTL clones require exogenous recombinant human interleukin-2 (rhIL-2) to be effective in these model systems consistent with adoptive therapy models demonstrating that tumor clearance by CTL specific for tumor antigens recognized,by TCR require rhIL-2 support to maintain in vivo persistence (Greenberg, 1986).

T cells can now be rendered specific for CD19, a cell surface molecule present on malignant B cells (U.S. published patent application No. 2004/0126363 A1). CD19 is an attractive target as the vast majority of B-ALLs uniformly express CD19, while expression is absent in nonhematopoietic, myeloid, erythroid, T cells, and bone marrow stem cells (Hulkkonen et al., 2002; Echeverri et al., 2002; LeBien, 2000). Moreover, primary human CD8⁺ cytotoxic T cell clones expressing a CD-19 specific chimeric immunoreceptor can specifically recognize and lyse CD19⁺ leukemia/lymphoma cells adding credence to this immunobased therapy (Cooper et al., 2003). A major limitation to the use of engineered cytotoxic T cells to target CD19 is the limited in vivo survival of the modified T cells due to an immune response against the expressed transgenes (Cooper et al., 2003). One novel mechanism to avoiding T cell-mediated targeting of the CD19-specific cytotoxic T lymphocytes (CTL's) would be to further modify the T cells to prevent presentation of the immunogenic transgenes by interrupting presentation of the expressed transgenes by classical human leukocyte antigen (HLA) molecules. The classical HLA molecules function both as alloantigens to trigger immune recognition (graft rejection of allogeneic cells in unmatched transplant recipients) and as a platform to present self or foreign peptides that can be recognized by CD8⁺ and CD4⁺ T cells bearing clonotypic T cell receptors (TCR's) (Adams and Parham, 2001). It has been demonstrated that enforced expression of viral immune evasion genes can modulate immune recognition by blocking expression of classical HLA class I molecules (Berger et al., 2000; York et al., 1994).

Adoptive immunotherapy with tumor-specific T cells is an attractive approach to treating human malignancies that are resistant to conventional therapeutic approaches. However, the widespread application of T cell therapy has been limited by a paucity of tumor-associated antigens (TAA) recognized by endogenous T cells and the difficulty of generating patient-specific T cells. The immunotherapy program at City of Hope is investigating the safety and feasibility of using genetically modified T cells that have been rendered tumor-specific. While this application of gene therapy to immunotherapy has broadened the number of TAA recognized by T cells, there still remains a critical delay between patient enrollment and the infusion of the tumor-specific T cells. What is needed, but up to now have been unavailable, are antigen-specific T cells that can be pre-prepared and cryopreserved be readily infused in all patients with a given antigen⁺ tumor. Thus, it is an object of the present invention to generate such “universal” T cells in patients with B-lineage ALL, whose disease is unresponsive to conventional chemotherapy, and to use such “universal T cells for treating B-ALL.

### SUMMARY OF THE INVENTION

The present invention is directed to universal T cells and their use in treating diseases and other physiological conditions. More specifically, the present invention is directed to universal T cells and their use in treating treating B-lineage acute lymphoblastic leukemia (B-ALL) in particular and malignancy in general. The universal T cells contain (i) nucleic acid encoding a chimeric antigen receptor (CAR) to redirect their antigen specificity and effector function and (ii) nucleic acids encoding shRNA and/or siRNA molecules to down-regulate cell-surface expression of T cell classical HLA class I and/or II genes to avoid recognition by recipient T cells. The universal T cells may also contain a nucleic acid encoding a non-classical HLA gene, such as an HLA E gene to enforce expression of HLA E genes and/or an HLA G gene to enforce expression of HLA G genes, to avoid recognition by recipient NK cells. The universal T cells may further contain a nucleic acid encoding a selection-suicide gene. For treating B-ALL the CAR is CD19R which comprises a single-chain anti-CD19 mouse immunoglobulin variable fragment (scFv) extracellular domain that is, in turn, fused to the cytoplasmic domain of CD3-ζ. The CD19R CAR, when expressed on the surface of cytolytic T lymphocytes (CTLs), re-directs their antigen specificity and effector function to CD19⁺ tumor cells, independent of classical HLA molecules.

Thus, in one aspect, the present invention provides universal T cells that have been genetically modified such that their antigen specificity and effector function have been re-directed to CD19⁺ tumor cells independent of classical HLA molecules. In one embodiment, the genetic modification of T cells is accomplished by the introduction of a nucleic acid encoding a CD19⁺ CAR into T cells. In one embodiment, the CD19⁺ CAR, also termed CD19R, comprises a single-chain anti-CD19 mouse immunoglobulin variable fragment (scFv) extracellular domain that is, in turn, fused to the cytoplasmic domain of CD3-ζ. In one embodiment, a nucleic acid encoding a CD19⁺ CAR is disclosed in U.S. published patent application No. 2004/0126363 A1, incorporated herein by reference. The T cells have also been modified to contain nucleic acids encoding shRNAs and/or siRNAs for modifying expression of HLA genes to avoid recognition by recipient T cells. In one embodiment, the shRNAs and/or siRNAs are used to achieye an enhanced siRNA effect, i.e., an enhanced down-regulation of cell-surface expression of T cell classical HLA class I and/or II genes. The universal T cells may also contain a nucleic acid encoding a non-classical HLA gene such as an HLA E gene to enforce expression of HLA E genes and/or an HLA G gene to enforce expression of HLA G, to avoid recognition by recipient NK cells. The T cells may also be further modified to contain a nucleic acid encoding a selection-suicide fusion protein, such as HyTK.

In a second aspect, the present invention provides a method for preparing the universal T cells. In one embodiment, the universal T cells are prepared by genetically modifying T cells using a non-viral electrotransfer system by which human T cells are genetically modified with plasmid vectors for co-expression of CD19R, siRNA, optionally non-classical HLA molecules, such as HLA E genes and/or HLA G genes, and optionally a selection-suicide fusion protein, such as HyTK. T cell products with chromosomally integrated plasmid vector are isolated and readily propagated to numbers in excess of 10¹⁰.

In a third aspect, the present invention provides a method for treating B-ALL which comprises administering a therapeutically effective amount of the universal T cells to individuals in need of such treatment.

### BRIEF DESCRIPTION OF THE FIGURES

FIGS. 1A and 1B show a schematic of the CD19R and plasmid. FIG. 1A: A schematic of the DNA plasmid CD19R/HyTK-pMG used to genetically modify T cells. The CD19R gene is under control of the human EF1α hybrid promoter. The HyTK gene is under control of the CMV promoter. The EM7 promoter is used to control the prokaryotic expression of hygromycin. The SV40 poly A site is 3′ of the CD19R gene and the bovine growth hormone polyA site is 3′ of the HyTK gene. FIG. 1B: A schematic of CD19R, ascFvFc:ζ chimeric immunoreceptor, composed of scFv, IgG4 hinge-Fc region, CD4 transmembrane region and CD3-ζ domain. The expressed receptor is shown as a dimer due to self-association of the C_{H}2 and C_{H}3 regions. Cell surface expression can be detected with Ab specific for human Fc region.

FIG. 2 shows an outline of manufacturing and quality control testing to produce universal CD19-specific T cells from umbilical cord blood.

FIG. 3 shows immunotherapy of Daudi tumor by CD19-specific UCBT. On day 0, 5×10⁶ ffLuc⁺ Daudi cells were subcutaneously injected in the left flank to three groups of NOD/scid mice. 50×10⁶ CD8⁺ CD19-specific T cells were given by tail-vein injection 10 days after implantation of subcutaneous ffluc⁺ Daudi tumor. Top images are prior to adoptive immunotherapy. Bottom images are after adoptive transfer. For anatomical localization, a pseudocolor image representing light intensity was generated in “Living Image” and superimposed over the grayscale reference image.

FIGS. 4A-4D show detection by chromium release assay (CRA) of a host cellular immune response against an infused T cell clone that expresses the neomycin (NeoR) phosphotransferase gene. T cells obtained pre-treatment (FIG. 4A and FIG. 4C) and 100 days after T cell infusion (FIG. 4B and FIG. 4D) are co-cultured ex vivo for 3 weeks with the infuised T cell clone (FIG. 4A and FIG. 4B) or autologous LCL (FIG. 4C and FIG. 4D). Targets for 4-hour CRA are autologous LCL, autologous LCL expressing Neo and the infused T cell clone.

FIG. 5 shows the sleeping beauty transposons system. The transposase and Transposon with therapeutic gene flanked by the inverted repeats are shown. Upon transfection the transposase is expressed and binds the inverted repeats flanking the gene of interest in the transposons and integrates the transposon into the target cells chromatin subsequently allowing the therapeutic gene expression from the context of the cellular genome.

FIG. 6 shows sleeping beauty transduced 293FT cells. SB-transposase (pCSB11) and SB transposons (pT2/BHEGFP, containing the EGFP transgene expressed from the CMV promoter) were EGFP⁺ relative to the negative control SB-transposase transfected cultures.

FIGS. 7A-7C show a system to titrate/augment expression of shRNA for down regulating HLA molecules using a plasmid vector. FIG. 7A: HLA ABC-specific (SEQ ID NO:1) or HLA A-specific (SEQ ID NO:2) U6shRNA cassette. The 9 nucleotide hairpin loops and 6 nucleotide terminator sequences are shown in lower case. The scrambled stem-loop is SEQ ID NO:3. FIG. 7B: Schematic of DNA expression plasmids EGFP/Neo-diipMG and HyTK-pMG, modified to express multiple copies of the U6shRNA cassettes. The EGFP gene is expressed from the human EF1 α promoter and NeoR or HyTK genes are expressed from the CMV IE promoter. Bovine growth hormone (bGhpA), late SV40 poly A (SV40pA), a synthetic poly A and pause site (SpAn), and E. coli origin of replication are also shown. FIG. 7C: HLA A3 molecule and relative binding sites of siRNA antisense strand and PCR primers. Signal peptide (sp) α1, 2, and 3 regions and cytoplasmic region are shown as determined from SWISSPROT: 1A03_HUMAN.

FIGS. 8A-8D show down regulation of HLA class I protein expression. FIG. 8A: Kinetic analysis of down regulation of HLA class I protein expression from multiple copies of the U6shRNA cassettes. Transfected Jurkat cells were analyzed for 5 days and RNAi activity represented by the percentage loss of binding of PE-conjugated anti-HLA ABC. FIG. 8B: Expression of multiple copies of the U6shRNA cassettes results in durable down regulation of classical HLA class I protein expression. G418-resistant Jurkat cells transfected with EGFP/Neo-diipMG plasmid with 0 to 8 copies of the U6shRNA cassette were analyzed by multiparameter flow cytometry for binding of PE-conjugated anti-β2m (x-axis) and CyChrome-conjugated anti-HLA ABC (y-axis), non-covalently expressed with soluble β₂-microglobulin on the cell surface, on EGFP⁺ cells. The binding of isotype control mAbs is shown. The percentage of cells in the lower left quadrant (HLA ABClowβ₂m^{low}) is shown for each plot. FIG. 8C: Southern blot analysis demonstrating integration of plasmids bearing U6shRNA cassettes. G418-resistant genetically modified Jurkat cells transfected with up to 8 copies of the anti- HLA ABC U6shRNA cassette. U6shRNA cassette copy number is indicated. FIG. 8D: Northern blot analysis of siRNA. Expression levels of shRNA in G418-resistant genetically modified Jurkat cells transfected with up to 8 copies of the U6 promoter and HLA ABC-specific shRNA, probed using an oligonucleotide complementary to the antisense strand of the shRNA. An oligonucleotide complementary to the endogenous U6 small nuclear (sn) RNA was used as an internal RNA loading standard. The U6shRNA cassette copy numbers are indicated.

FIG. 9 shows phenotypic effects of HLA A-specific siRNA in differentiated primary human T cells. Down-regulation of cell-surface HLA A2 (and HLA ABC, insert) protein expression on hygromycin-resistant heterozygous (donor #1, HLA A*0201/0301, B*0702/1402) or homozygous (donor #2, HLA A*0201/0201, B*0702/3503) HLA A2⁺ primary T cells transfected with a HyTK-pMG DNA plasmid modified to express 6 copies of the shRNA cassette. T cells were analyzed by flow cytometry for bindin of PE-conjugated anti-HLA A2 and HLA ABC. Dead cells were excluded by uptake of PI.

FIG. 10 shows sets of SB plasmids to be used in transfection efficiency experiments. Set 1: the most basic constituents of the SB system which consists of the SB-transposase and one SB-transposon with EGFP-Neomycin fusion expressed from the CMV pol-II promoter shown as filled triangle. Set 2: an expanded SB system to look at the efficiency of the SB-transposase to effectively integrate 2 SB-transposons, SB-Transposon (A) from Set 1 and SB-Transposon (B) expressing dsRED-HyTK fusion expressed from a pol-II promoter. Set 3: a non-drug selected transposon system utilizing the SB-Transposase and an SB-Transposon (C) expressing CD19R-HyTK fusion cassette. Set 4: an expanded transposon system consisting of SB-Transposase and SB-Transposon (D) expressing HLA-E, shRNAs targeting HLA-II (mRNA specific), an siRNA targeting HLA-I (promoter specific) and the Neomycin suicide gene cotransfected with SB-Transposon (E) expressing shRNAs targeting HLA-I (mRNA specific), siRNA targeting HLA-II (promoter specific) and CD19R fused to the Hy-TK selection/suicide gene (SG). The shRNA and siRNAs are expressed from (U6) Pol-III promoters shown as open triangles while the filled triangles represent Pol-II promoters.

FIG. 11 shows a schematic of gene transfer using the sleeping beauty (SB) transposon system in a three-plasmid transfection. Specifically Set 4 described in FIG. 10 consisting of a SB-Transposase (modified for enhanced integration of larger transposons (Yant et al., 2004) and Table 3) and two SB-Transposons, SB-Transposon (D in Set 4) expressing HLA-E, shRNAs targeting HLA-II (mRNA specific), siRNA targeting HLA-I (promoter specific) and Neomycin suicide gene, and SB-Transposon (E in Set 4) expressing shRNAs targeting HLA-I (mRNA specific), siRNA targeting HLA-II (promoter specific) and CD19R fused to the Hy-TK suicide/selection gene (SG) is used. The suicide genes are necessary to allow cells harboring the therapeutic transgenes to be killed if the transposon is turning on undesirable genes. The shRNAs and siRNAs are expressed from (U6) Pol-III promoters shown as open triangles while the filled triangles represent Pol-II promoters. The SB-Transposase and SB-Transposons (D in Set 4) and (E in Set 4) are co-transfected (Amaxa NucleofectorTM) into CD8⁺ T cells (1). Following co-transfection the SB-Transposase enzyme is expressed (2) and binds to the inverted repeats (IR) in the SB-Transposon (3). The SB-transposase bound transposons are then directly integrated into the target host cell chromatin by the SB-transposase producing a stable transduced CD8⁺ T cell (4). The IRs for SB-Transposon D in Set 4 are located on the right in (4) and the IRs for SB-Transposon E in Set 4 are located on the left in (4).

FIG. 12 shows inter-patient dose escalation and de-escalation is dependent of monitoring of dose limiting toxicities (DLT). Dose escalation is permnitted, if after 28 days of a T cell infusion, less than two of the three research participants for a given Dose Level has not developed a new adverse event of grade ≧3 involving GVHD, cardiopulmonary, hepatic (excluding albumin), neurologic, or renal CTC vs. 3 parameters that is probably or definitely attributed to the infused T cell product. Should Adverse Events/toxicities be observed that result in cessation of treatment of patients at that dose-level/result in failure to met criteria for cohort dose escalation, three additional patients will be treated at the prior Dose Level.

FIG. 13 shows a timeline depicting time points for immunocorrelative studies up to 100 days after infusion of universal Cd19-specific T cells. Day 0 is defined as day the 1/10^{th} T cell dose is infused. Up to 40 mL peripheral blood and 15 mL bone marrow (maximum 1.5 mL/Kg) to be removed at each time point.

### DETAILED DESCRIPTION OF THE INVENTION

The present invention is directed to universal T cells and their use in treating diseases and other physiological conditions. More specifically, the present invention is directed to universal T cells and their use in treating treating B-lineage acute lymphoblastic leukemia (B-ALL) in particular and malignancy in general. The universal T cells contain (i) nucleic acid encoding a chimeric antigen receptor (CAR) to redirect their antigen specificity and effector function and (ii) nucleic acids encoding shRNA and/or siRNA molecules to down-regulate cell-surface expression of T cell classical HLA class I and/or II genes to avoid recognition by recipient T cells. The universal T cells may also contain a nucleic acid encoding a non-classical HLA E gene to enforce expression of HLA E genes to avoid recognition by recipient NK cells. The universal T cells may further contain a nucleic acid encoding a selection-suicide gene. For treating B-ALL the CAR is CD19R which comprises a single-chain anti-CD19 mouse immunoglobulin variable fragment (scFv) extracellular domain that is, in turn, fused to the cytoplasmic domain of CD3-ζ. The CD19R CAR, when expressed on the surface of cytolytic T lymphocytes (CTLs), re-directs their antigen specificity and effector function to CD19⁺ tumor cells, independent of classical HLA molecules.

Thus, in one aspect, the present invention provides universal T cells that have been genetically modified such that their antigen specificity and effector function have been re-directed to CD19⁺ tumor cells independent of classical HLA molecules. In one embodiment, the genetic modification of T cells is accomplished by the introduction of a nucleic acid encoding a CD19⁺ CAR into T cells. In one embodiment, the CD19⁺ CAR, also termed CD19R, comprises a single-chain anti-CD19 mouse immunoglobulin variable fragment (scFv) extracellular domain that is, in turn, fused to the cytoplasmic domain of CD3-ζ. In one embodiment, a nucleic acid encoding a CD19⁺ CAR is disclosed in U.S. published patent application No. 2004/0126363 A1 and in PCT international published patent application No. WO 02/77029, each incorporated herein by reference. In one embodiment, the CAR is operably linked to a promoter. As used herein, components of a construct referred to as being operably linked or operatively linked refer to components being so connected as to allow them to function together for their intended purpose. For example, a promoter and a coding region are operably linked if the promoter can function to result in transcription of the coding region. Any suitable promoter well known in the art can be used to drive expression of the CAR. In one embodiment, the promoter is the human EF1 α hybrid promoter (Kim et al. (1990).

The T cells are further modified such that RNA interference (RNAi) is used to specifically target and suppress HLA class I and/or II expression to avoid immune recognition and subsequent destruction of the infused therapeutic T cell by the recipient's own T cells.

RNAi is a process in which double-stranded RNA induces homology dependent degradation of mRNA (Montgomery et al., 1998; Mishikura, 2001; Sharp, 2001). RNAi can suppress gene expression via two distinct pathways: transcriptional (TGS) and post-transcriptional (PTGS) gene silencing (Sijen et al., 2001; Pal-Bhadra et al., 2002). PTGS involves small interfering RNAs (shRNAs) targeting of either mRNA or pre-mRNA, including intronic sequences in C. elegans and yeast (Bosher et al., 1999) (reviewed (Ramaswamy and Slack, 2002)). Conversely, TGS was first described in virus-infected plants, which contained promoters with homology to the viral sequences. These promoters became methylated at sites matching the small double stranded viral siRNAs and transcription suppressed as a result of these homologous viral RNAs entering the nucleus and inducing TGS (Wassenegger, 2000; Wassenegger et al., 1994), i.e RNA-specific promoter targeted suppression. In human cells, gene silencing induced by RNAi was initially thought to be restricted to action on cytoplasmic mRNA or RNA at the nuclear pore (Zeng and Cullen, 2002), similar to most reports in C. elegans and T brucei (Montgomery et al., 1998; Fire et al., 1998; Ngo et al., 1998). To date, TGS has been found to occur in plants, Drosophila, and in S. pombe in centromeric regulation (Volpe et al., 2002), while non RNAi mediated TGS has been documented in Rat fibroblasts (Bahramian and Zarbul, 1999). Recently, it was observed that small interfering RNAs directed against the elongation factor 1 alpha promoter (EF1α) can direct TGS in human cells and that this phenomena relied on direct nuclear delivery of the siRNA (Morris et al, 2004). Moreover, the observed inhibition of expression was reversible with the addition of 5-azactadine (5-Aza C, 4μM) and trichostatin A (TSA, 0.05mM) to the transduced siRNA or MPG/siRNA transfected cultures and was associated with promoter specific methylation, suggesting siRNA induced TGS in human cells is also linked to histone modifications. In accordance with the present invention, RNAi is applied to a therapeutically applicable target for the construction of a universal donor T cell population with modified expression of HLA genes to treat B-ALL.

The Major Histocompatibility Complex (MHC) is one of the most gene dense regions in the human genome (Marsh et al., 2002). Two families of genes in the MHC (class I and class II) encode highly polymorphic Human Leukocyte Antigens (HLA) that are involved in antigen presentation. Three classical class I genes (HLA-A, -B and -C) are typically expressed on the surface of most nucleated cells in the body and are recognized by two distinct cytolytic lymphocytes: cytotoxic T cells (CTL) and natural killer (NK) cells. The effector cytotoxicity and cytokine secretion functions of NK cells are controlled by two distinct sets of HLA class I-specific receptors: activating NK receptors and inhibitory NK receptors (Lanier, 1998). A fine balance between these two types of HLA class I-specific receptors controls NK cell function. Binding of HLA class I and specific inhibitory NK receptors generates a dominant inhibitory signal that neutralizes any positive signals in the NK cells, and thereby the self class I protects healthy cells from the NK lysis (Lanier, 1998; Ljunggren and Karre, 1990). This mechanism prevents NK cells from attacking healthy autologous cells and directs them to kill cells with impaired expression of MHC class I, as can occur during viral infection and progressive tumor growth. Human NK cells express two structurally distinct families of MHC class I receptors: killer cell immunoglobulin-like receptors (KIR) and lectin-like receptors. The former are receptors are specific to polymorphic classical class I HLA molecules. The latter are expressed either as heterodimers (CD94:NKG2), specific to HLA-E (a non-classical MHC class I) or homodimers (NKG2D:NKG2D), which recognize a variety of ligands having MHC class I-like structure (including MICA and MICB).

In one embodiment, the T cells are modified to contain a siRNA construct targeting HLA class I genes. In another embodiment, the T cells are modified to contain a siRNA construct targeting HLA class II genes. In a further embodiment, the T cells are modified to contain a siRNA construct targeting HLA class I genes and a siRNA construct targeting HLA class II genes. In one embodiment, the T cells are modified to contain a shRNA construct targeting HLA class I genes. In another embodiment, the T cells are modified to contain a shRNA construct targeting HLA class II genes. In a further embodiment, the T cells are modified to contain a shRNA construct targeting HLA class I genes and a shRNA construct targeting HLA class II genes. In one embodiment, the T cells are modified to contain a shRNA construct targeting HLA class I genes and a siRNA construct targeting HLA class I genes. In another embodiment, the T cells are modified to contain a shRNA construct targeting HLA class II genes and a siRNA construct targeting HLA class II genes. In a further embodiment, the T cells are modified to contain a shRNA construct targeting HLA class I genes, a siRNA construct targeting HLA class I genes, a shRNA construct targeting HLA class II genes and a siRNA construct targeting HLA class I genes. The siRNA and shRNA constructs are designed and prepared using techniques well known in the art.

The T cells may also be modified to contain a nucleic acid encoding a non-classical HLA gene. The non-classical HLA gene may be an HLA E gene to enforce expression of HLA E genes to avoid recognition of the universal T cells by the recipient's own NK cells. The non-classical HLA gene may be an HLA G gene to enforce expression of HLA G genes to avoid recognition of the universal T cells by the recipient's own NK cells. The non-classical HLA gene may be both an HLA E gene and an HLA G gene. In one embodiment, the HLA E gene is a chimeric gene which uses the HLA-A2 signal sequence to achieve surface expression. In one embodiment, a nucleic acid encoding the HLA E chimeric gene is disclosed in Lee et al. (1998), incorporated herein by reference. In one embodiment, this coding sequence is further modified by introducing conservative point mutations that do not affect the coding capacity of the chimeric HLA E gene, but elude MRNA degradation by the same shRNAs that target the HLA class I genes. In another embodiment, the coding sequence is further modified to contain a FLAG-tag so that chimeric HLA E protein can be distinguished from the endogenous HLA E protein. The coding sequence is operably linked to a promoter. Any suitable promoter well known in the art can be used to drive expression of the chimeric HLA E. In one embodiment, the promoter is a strong promoter. In one embodiment, the strong promoter is a Pol-II viral promoter, which avoids down regulation of the endogenous promoter driving HLA E expression.

The T cells may be further modified to contain a nucleic acid encoding a selection-suicide gene. In one embodiment, the selection-suicide gene encodes the fusion protein HyTK. HyTK directs the synthesis of a bifunctional fusion protein incorporating hygromycin phosphotransferase and herpes virus thymidine kinase (HSV-TK) permitting in vitro selection with hygromycin and in vivo ablation of transfected cells with gancyclovir. In one embodiment a nucleic acid encoding HyTK is disclosed in Lupton et al. (1991), incorporated herein by reference. In one embodiment, the coding sequence is operably linked to a promoter. Any suitable promoter well known in the art can be used to drive expression of the selection-suicide fusion protein. In one embodiment, the coding sequence is fused in frame with the coding sequence of the CAR.

T cells are obtained from any appropriate source. In one embodiment, T cells are obtained from umbilical cord blood. Umbilical cord blood T cells (UCBT) are particularly useful because of two properties intrinsic to UCBT: (i) The increased replicative potential of UCBT, as demonstrated by their greater telomere length, relative to T cells derived from peripheral blood (Li et al., 1994; Mackall et al., 1997) which translates into improved rates of ex vivo expansion and decreased probability for replication senescence in vivo after adoptive transfer and (ii) transplanted umbilical cord blood T cells have a higher tolerance to human leukocyte antigen (HLA) mismatch (Li et al., 1994; Mackall et al., 1997).

Universal T cells containing the nucleic acids described above are prepared using conventional techniques for introducing nucleic acids into cells. The term “introducing” encompasses a variety of methods of introducing DNA into a cell, either in vitro or in vivo. Such methods include transformation, transduction, transfection, and infection. The introducing may be accomplished using at one or more vectors, which include plasmid vectors and viral vectors. Viral vectors include retroviral vectors, lentiviral vectors, or other vectors such as adenoviral vectors or adeno-associated vectors. Alternate delivery of nucleic acids into cells or tissues may also be used in the present invention, including liposomes, chemical solvents, electroporation, transposons, as well as other delivery systems known in the art. Thus, in one embodiment, the nucleic acids are introduced into T cells by viral, e.g., retroviral, gene transfer according to techniques well known in the art. In another embodiment, the nucleic acids are introduced into T cells by non-viral gene transfer according to techniques well known in the art. In a further embodiment, the nucleic acids are introduced into T cells using a transposon system according to techniques well known in the art and as described herein. In one embodiment, the transposon system is the sleeping beauty (SB) transposon system, which has been used to transfer nucleic acids into human cells (Liu et al., 2004; Geurts et al., 2003; Izsvak et al., 2000).

In this embodiment, an SB transposase is used to introduce the nucleic acids of the present invention into T cells. In one embodiment, an SB transposase protein is introduced into T cells. In another embodiment, an RNA encoding an SB transposase is introduced into T cells. In accordance with this embodiment, an SB transposase transcript may be synthesized in vitro or isolated from a biological source. In one aspect, a nucleic acid construct is prepared which contains an RNA polymerase promoter and the coding sequence for an SB transposase. The RNA polymerase promoter is preferably the SP6 promoter. However, other RNA polymerase promoters can be used, including the T7 promoter. The nucleic acid construct further comprises 5′- and 3′-UTRs and a polyA tail. Any 5′- and 3′-UTRs and any polyA tail may be used. In a further embodiment, a vector containing a nucleic acid encoding an SB transposase is introduced into T cells. The nucleic acid encoding an SB transposase is operably linked to a promoter. In one aspect, the promoter is an RNA polymerase promoter, such as described above. In another aspect, the promoter is a sequence or sequences of DNA that function when in a relatively fixed location in regard to the transcription start site. A promoter contains core elements required for basic interaction of RNA polymerase and transcription factors, and may contain upstream elements and response elements. Any suitable promoter, such as CMV pol-II promoter and other promoters well known in the art, can be used to drive expression of an SB transposase.

In accordance with the present invention, the SB transposase is used to cause the transposition of the nucleic acids of the present invention from vectors that contain one or more of the nucleic acids of the present invention into the genome of the T cells. Vectors containing the nucleic acids of the present invention art also termed SB transposons herein. The one or more nucleic acids of the present invention are positioned in the vectors between internal repeats recognized by the SB transposase. Any suitable vector, e.g., a plasmid vector, a viral vector, and the like can be used as an SB transposon. In one embodiment, an SB transposon contains a single nucleic acid of the present invention. In another embodiment, an SB transposon contains two nucleic acids of the present invention. In a further embodiment, an SB transposon contains three nucleic acids of the present invention. In a still further embodiment, an SB transposon contains more than three nucleic acids of the present invention. Each nucleic acid is under the control of an appropriate promoter as described above. In one embodiment, the nucleic acids encoding the CAR and the non classical HLA gene, such as HLA E gene and/or HLA G gene, are under control of a promoter that contains core elements required for basic interaction of RNA polymerase and transcription factors, and may contain upstream elements and response elements. Any suitable promoter, such as CMV pol-II promoter and other promoters well known in the art, can be used. In one embodiment, the nucleic acids encoding the shRNAs and siRNAs are under control of an RNA polymerase promoter, such as a U6 pol-III promoter (U6 small nuclear RNA promoter (Lee et al., 2002)) and other promoters well known in the art.

In accordance with the present invention, an SB transposase and one or more SB transposons are introduced into T cells. The SB transposase is introduced into T cells as described above. In one embodiment, a three vector system is used to prepare the universal T cells of the present invention. One vector contains a nucleic acid encoding an SB transposase under control of an appropriate promoter. A second vector contains a nucleic acid encoding an shRNA for HLA class I, a nucleic acid encoding an siRNA for HLA class II and a nucleic acid encoding a CAR. In one embodiment, the CAR is CD19R. In one embodiment, the nucleic acid encoding the CAR further includes a nucleic acid encoding a selection-suicide gene in frame with the nucleic acid encoding the CAR. A third vector contains a nucleic acid a non-classical HLA gene, such as an HLA E gene described herein and/or an HLA-G gene, a nucleic acid encoding an shRNA for HLA class II and a nucleic acid encoding an siRNA for HLA class I. The third vector may further contain a marker gene. Alternatively, if both an HLA E gene and an HLA G gene are used, they may be located on separate vectors. The three vectors are introduced into T cells using conventional techniques well known in the art, such as electroporation, or the techniques described herein.

Following transfection, T cells with cell surface expression of CAR are isolated. In one embodiment, T cells with cell surface expression of CD19R are isolated and rapidly expanded with OKT3 and IL-2 in accordance with conventional techniques or as described herein. At the end of the second 14-day growth cycle (mediated by OKT3) the cell surface expression of the CD19R are assessed using anti-CD19 and anti-FC. The T cells with cell surface expression of CD19R are then analyzed for down regulation of the HLA class I and/or class II genes, and optionally for expression of the non-classical HLA genes. These analyses are performed using conventional techniques well known to a skilled artisan or those described herein. The universal T cells of the present invention are subjected to recursive 14 day expansion cycles, after which banks of about 10¹¹ universal T cells are cryopreserved. Aliquots of these universal T cells are used for treating patients with B-ALL.

Patients can be treated by infusing therapeutically effective doses of CD8⁺ universal T cells in the range of about 10⁶ to 10¹⁰ or more cells per square meter of body surface (cells/m²). The infusion is repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/m² are infused, escalating to 10¹⁰ or more celis/m². IL-2, e.g., rhIL-2, can be co-administered to expand infused cells post-infusion. The amount of IL-2 can be about 10⁵ to 10⁶ units per square meter of body surface per dose. Doses may be administered every 12 hours.

In similar manner, the concept of producing cells with loss of classical HLA expression may broaden the application of cellular therapy in general. For example, stem cells, such as but not limited to, embryonic stem cells, hematopoietic stem cells, pancreatic stem cells, could be genetically modified to down regulate expression of HLA molecules. This genetically modified biologic material might be infused in recipients regardless of HLA background or matching and the loss of HLA expression in the infused material would help avoid immune-mediated rejection of the transplanted cellular material and/or the need for the recipient to receive immunosuppression to prevent this immune mediated rejection. Furthermore, the down-regulation of HLA molecules would precule development of an immune reposne against immunogeneic transgenes which might be expressed in the cellular agents.

In a similar manner, universal T cells are prepared with (i) re-directed specificity for CD20 (for CD20-specific re-directed T cells see U.S. Pat. No. 6,410,319, incorporated herein by reference), CE7 (for CE7-specific re-directed T cells see U.S. published patent application No. 2003/0215427 A1, incorporated herein by reference) or receptor ligands, such as those involved with cancer (for such specific re-directed T cells see U.S. published patent application No. 2003/0171546 A1), (ii) modified HLA class I and/or II gene expression, optionally (iii) enforced HLA E expression and optionally (iv) a selection-suicide gene. These universal T cells are used to treat diseases or conditions such as those described in the cited patents or published applications in a manner similar to that described herein.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); Jakoby and Pastan (eds.), Cell Culture. Methods in Enzymology, Vol. 58 (Academic Press, Inc., Harcourt Brace Jovanovich, N.Y., 1979).; Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

### EXAMPLES

The present invention is described by reference to the following Examples, which are offered by way of illustration and are not intended to limit the invention in any manner. Standard techniques well known in the art or the techniques specifically described below were utilized.

### Example 1

### Ex vivo Isolation and Expansion of Universal CD19-Specific Umbilical Cord Blood-Derived T Cells

The decision to use umbilical cord blood T cells (UCBT) as a platform for genetic modification and preparation of universal T cells is based on two properties intrinsic to UCBT: (i) The increased replicative potential of UCBT, as demonstrated by their greater telomere length, relative to T cells derived from peripheral blood (Li et al., 1994; Mackall et al., 1997) which translates into improved rates of ex vivo expansion and decreased probability for replication senescence in vivo after adoptive transfer and (ii) transplanted umbilical cord blood T cells have a higher tolerance to human leukocyte antigen (HLA) mismatch (Li et al., 1994; Mackall et al., 1997), which may reduce the potential for deleterious recognition of allo-antigens by the endogenous T cell receptor (TCR) expressed on the infused universal T cells. This is demonstrated by the low risk of graft-versus-host disease (GVHD) in patients undergoing allogeneic umbilical cord blood transplantation (Li et al., 1994; Mackall et al., 1997). Various immunologic properties of cord blood are thought contribute to the reduction of GVHD after umbilical cord blood transplantation. In particular, it is known that cord blood T cells are functionally naive lymphocytes (Li et al., 1994; Mackall et al., 1997; Goldrath and Bevan, 1999; Sprent and Surh, 2003; Marrack et al., 2000), and in particular exhibit markedly reduced responsiveness in vitro to allogeneic stimuli in secondary mixed lymphocyte reaction. This unresponsiveness to secondary stimulation occurs in spite of TCR and co-stimulatory activation (Li et al., 1994; Mackall et al., 1997).

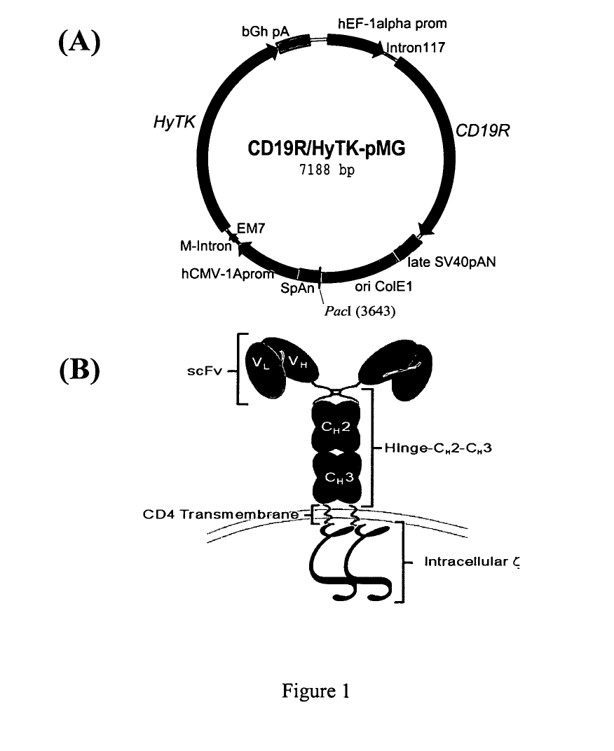

To target B-ALL, a CD19-specific chimeric immunoreceptor, designated CD19R, has been generated that combines antibody recognition with T cell effector functions. The specificity of CD19R is derived from the variable regions of a mouse monoclonal antibody (mAb) specific for CD19 that are tethered to the T cell via a modified human IgG4 hinge/Fc region and CD4 transmembrane domain (FIG. 1). Upon binding CD19, the genetically modified T cells are activated by the cytoplasmic CD3-ζ chain fused to the immunoreceptor (Li et al., 1994).

To avoid T cell-mediated clearance of adoptively transferred genetically modified UCBT, plasmid vectors which down regulate surface expression of classical HLA-I and HLA-II molecules are constructed and tested. The primary approach is to use siRNA and shRNA molecules to disrupt classical HLA gene expression by targeting mRNA and promoter. Furthermore, to avoid NK-T-mediated clearance of infused T cells which are HLAI/II^{null}, the plasmid vectors co-express non-classical HLA genes, which serve as inhibitory ligands for the killer cell immunoglobulin-like receptor (KIR) family of receptors on NK cells.

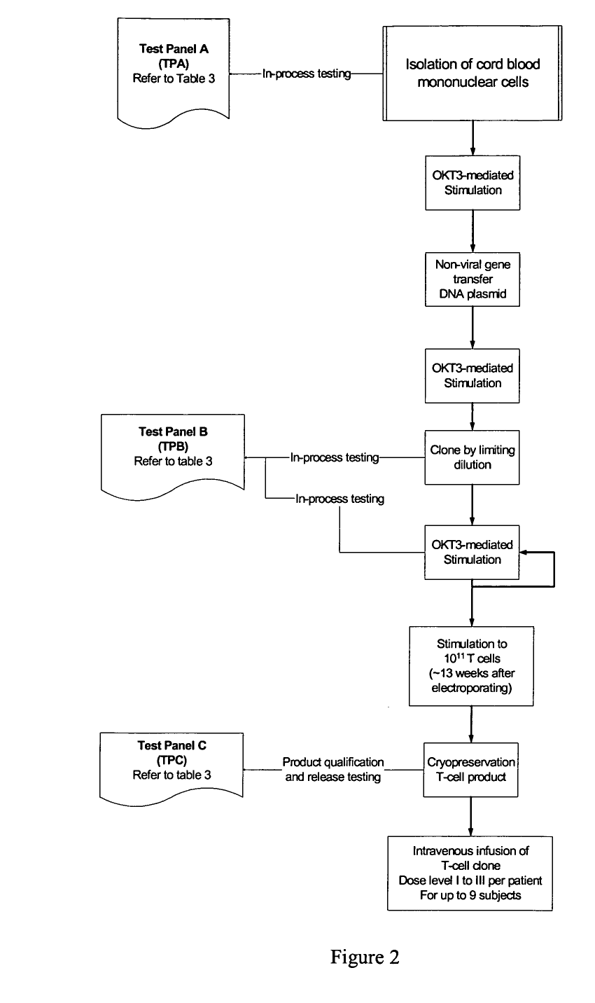

Non-viral gene transfer has been developed as a methodology to successfully genetically modify human cord blood-derived T cells for clinical trials (FIG. 2). Briefly, after cord blood WBC were purified by Ficoll-Hypaque gradient and stimulated with OKT3 (30 ng/mL) and beginning on the first day of culture, recombinant human interleukin-2 (rhIL-2) was added (25 IU/mL) every-other-day. On the third day of culture, the cells are resuspended in hypotonic electroporation buffer (Eppendorf, Hamburg, Germany) at up to 20×10⁶/mL and 400 μL of the cell culture are aliquoted into sterile electroporation cuvettes and electroporated with a single electrical pulse of with 7.5 μg linearized plasmid, e.g. CD19R/HyTK-pMG plasmid DNA (FIG. 1) which is currently being manufactured under FDA masterfile BB-MF978 for use in a clinical trial. This plasmid co-expresses the bi-functional hygromycin phosphotransferase/thymidine kinase selection/suicide gene (HyTK), which permits in vitro selection with hygromycin B, and potential in vivo ablation using ganciclovir. On the fifth day of culture cytocidal concentrations of hygromycin B were added. After 14 days in culture the UCBT are cloned by limiting dilution by seeding T cells into 10 sets of 96-well plates at densities of 5000 cells/well, 2,500 cells/well, 1,250 cells/well, 625 cells/well. Each well contains 200 μL media supplemented with OKT3, rhIL-2 50 U/mL, thawed 10⁵ γ-irradiated PBMC (obtained from healthy donors and cryopreserved in compliance with cGMP) and 2×10⁴ γ-irradiated TM-LCL (obtained from a master-cell bank prepared in compliance with cGMP under BB-IND 11411) per well. After 5 days, cytocidal concentrations of hygromycin B and rhIL-2 (50 U/mL) are added at to the plates. After 21 days of culture, plates are selected in which wells demonstrating growth are <30%. 20 to 30 T cell clones are selected that lyse CD19⁺ targets by high-throughput micro-CRA and are CD3⁺ by micro-flow cytometry.

Based on the T cell rapid expansion protocol developed by Riddell and Greenberg (1990), the clones are expanded with OKT3, thawed 50×10⁶ γ-irradiated PBMC and 10⁷ γ-irradiated TM-LCL. Beginning on day 1, rhIL-2 is added at 50 U/mL and replenished every 48 hours. Cytocidal concentrations of hygromycin B are added beginning on day 5. The 14-day stimulation cycles are repeated to obtain sufficient CD19-specific T cells for in-process testing (Test Panel B in FIG. 2 and Table 1) to validate that chimeric CD3-ζ is expressed and that the T cells exhibit CD19-specific re-directed lysis. The clones can also be ranked by their ability to numerically expand in vitro, their relative ability to lyse CD19⁺ targets and their relative telomere length using a fluorescence-based in situ telemere-length assay (Flow-FISH) (Goulden et al., 2003; Philip and Biron, 1991). The top performing clones are then expanded to establish a cryopreserved cell bank.

TABLE 1
Release Criteria and In-Process Testing for Universal T Cell Product
Test Panel A (performed on frozen UCB)

| Test | Release Criteria | Test Method |
|---|---|---|
| Viability | ≧60% Viable | Trypan blue exclusion test |
| Sterility | Negative for bacteria at 14 days; Negative for fungus at | USP |
| | 28 days | |
| Mycoplasma Assay | Negative for mycoplasma | PCR |

Testo Panel B (performed on genetically modified T cells)

| Test | In Process Test | Test Method |
|---|---|---|
| CD19-specific | Top quartile specific lysis again 2000 cells of CD19+ cell | 4 hr-micro-Chromium |
| cytolytic activity | line | release assay |
| Telomere length | Top quartile longest telomere fluorescence | FISH-Flow (molecular |
| | | equivalents of soluble |
| | | fluorochrome units) |
| Chimeric: ζ receptor | 66-kD chimeric protein band | Western Blot with human |
| expression | | CD3ζ-specific |
| | | primary antibody |
| T cells surface | <5% HLA ABC⁻ <5% HLA DR⁻ ≧90% HLA E⁺ ≧90% | Flow cytometric evaluation |
| phenotype | CD 8⁺ ≧90% CD3⁺ | |

Test Panel C (performed on cryopreserved T cells bank)

| Test | Release Criteria | Test Method |
|---|---|---|
| Viability | ≧80% Viable | Trypan blue exclusion test |
| Sterility | Negative for bacteria at 14 days; Negative for fungus | USP |
| | at 28 days | |
| Sterility | Adventitial virus testing¹ | PCR |
| Mycoplasma assay | Negative for mycoplasma | PCR |
| Clonality | Single band using hygromycin-specific probe | Southern Blot |
| Chimeric: ζ receptor | 66-kD chimeric protein band | Western Blot with human |
| expression | | CD3ζ-specific |
| | | primary antibody |
| Sensitivity to | Cell Numbers ≦10% of positive control | 2 weeks continuous |
| ganciclovir-ablation | | culture in 5 μM ganciclovir |
| Dependence on | Cell Numbers ≦10% of positive control | 2 weeks continuous culture |
| rhIL-2 for growth | | without exogenous rhIL-2 |
| T cell surface | <5% HLA ABC⁻ <5% HLA DR⁻≧90% HLA E⁺ ≧90% | Flow cytometric evaluation |
| phenotype | CD8⁺ ≧90% CD3⁺ | |
| CD19-specific | ≧30% Specifgic Iysis at 50:1 (E:T) against a CD19⁺ | 4 hr-Chromium release |
| cytolytic activity | cell line | assay |
| γ-IFN Production | ≧200 pg/mL incubated 28-hrs at CD19⁺ stimulator: | Cytokine bead array |
| | responder cell ratio of 1:1 | |
| Plasmic integration | Presence of single band using hygromycin-specific | Southern Blot |
| | probe | |
| Endotoxin | Endotoxin burden <5 EIU/Kg recipient body | Chromogenic Limulus |
| | weight/hour of infusion² | Amebocyte Lysate (LAL) |
| Karyoptye | Normal | Cytogenetics |

¹HTLVI/II PCR, Hepatitis B PCR, CMV PCR, HCV RT-PCR, HIV-1/2 PCR
²Typical T-cell intravenous infusion time is over 30 minutes

### Example 2

### Umbilical Cord Blood-Derived T Cells Can Be Rendered Specific for CD19

Following expansion, genetically modified cord blood-derived T cells can be harvested and evaluated by Western blot for expression of the chimeric immunoreceptor protein by probing with an anti-ζ mAb. Unmodified and modified T cells display a 21-kDa band consistent with wild-type CD3-ζ chain, but genetically modified T cells demonstrate a second band of ˜66-kDa consistent with the chimeric-ζ chain. Flow cytometry was used to show that the expanded genetically modified T cell clones were typically CD8⁺TCRαβ⁺Fc⁺. The ability of the genetically modified CD19R⁺ T cells to lyse CD19⁺ targets was assessed by a 4-hour chromium release assay (CRA). CD19-specific CTL were able to lyse human tumor lines independent of HLA molecules if the targets expressed CD19, but were unable to lyse targets that were CD19⁻. To show that the genetically modified CTL were activated for cytokine production, the CD19R⁺ T cells were stimulated with CD19⁺ and CD19 ⁻lines and secretion of cytokines was quantified by ELISA or cytokine bead array (CBA). Only stimulators derived from human tumor lines expressing CD19 were able to activate CD19R⁺ T cells to produce γ-IFN. The sensitivity of the CD19R⁺HyTK⁺ T cells to TK-mediated ganciclovir (GCV) mediated-ablation can be demonstrated in vitro and in vivo. The function of genetically modified CD19R⁺ T cells has been studied in vivo using a xenogeneic mouse tumor model.

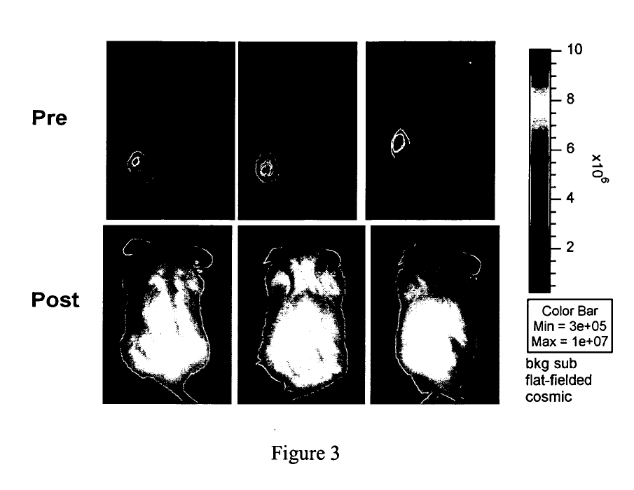

To non-invasively monitor the efficacy of immunotherapy using the Xenogen system, Daudi cells, derived from a human Burkitts lymphoma, (Foon et al., 1986) have been genetically modified to co-express firefly (Photinus pyralis) luciferase (ffluc) and the zeocin-resistance gene (Invitrogen). The ffLuc gene was adapted for eukaryotic expression and fusing the two genes in frame generated a luciferase-zeocin chimeric protein. We have demonstrated that NOD/scid mice bearing zeomycin-resistant ffLuc⁺Daudi cells could be treated with infusions of CD19-specific T cells (FIG. 3). These animal experiments correlated with studies showing that genetically modified T cells migrate along chemotaxis gradients established by tumors.

### Example 3

### Manufacturing and Infusing CAR Re-Directed CTL into Oncology Patients

Investigators at City of Hope have established technologies for the ex vivo genetic modification, cloning, and large-scale expansion of human T-lymphocytes for FDA-authorized clinical trials. City of Hope's cGMP-compliant biologics manufacturing facility—The Center for Biomedicine and Genetics (CBG)—is a licensed built-to-suit 20,000 ft² facility having three separate production areas for the manufacturing of viral vectors, recombinant protein and DNA, and ex vivo manipulated cell products. The CBG has established a FDA masterfile for plasmid DNA production (BB-MF#9778) and has recently been designated as a NGVL production site for clinical-grade plasmid DNA. A cell production suite within the CBG has been allocated for T cell manufacturing. These core technologies, and COH's infrastructure to support them, set the stage for the implementation of a series of rapidly deployed cellular immunotherapy clinical trials designed to delineate key biologic parameters pertaining to the interface of this class of therapeutics with the oncology patient population. These clinical studies provide information that will facilitate laboratory-based research efforts to further enhance the anti-tumor immunobiology of genetically modified T cells. COH has demonstrated its capacity to conduct research in this paradigm as evidenced by the rapid transition of preclinical studies to FDA-authorized adoptive therapy trials described below and supported to a significant degree by our General Clinical Research Center (GCRC).

These institutional resources have enabled us to commence with clinical pilot feasibility/safety trials utilizing genetically engineered autologous CTL clones. COHNMC IRB#98142 (BB-IND#8513) involving five enrolled research participants with recurrent CD20⁺ B-cell non-Hodgkin lymphoma was initiated. On this trial, patients underwent a series of three escalating cell doses (10⁸,10⁹,10¹⁰ clone/m²) of autologous CD8⁺ CTL-clones genetically modified to co-express CD20-specific scFvFc:ζ CAR and Neo^{R} genes, shortly following autologous HCT. This trial represented a strategic investment in the development of antigen-specific cellular immunotherapy for targeting post-transplant lymphoma minimal residual disease, the most common etiology of treatment failure in autologous stem cell transplantation for lymphoma. A second Pilot study was initiated in 2001 targeting neuroblastoma with clones co-expressing a L1-CAM-specific scFvFc:ζ receptor and the selection-suicide fusion protein HyTK (COHNMC IRB#99183/BB-IND#9149/PI).

The primary objective of this adoptive transfer study was to evaluate the feasibility and safety of escalating cell doses of autologous L1-CAM-specific CTL-clones. Secondary objectives were focused on gaining insights into the in vivo biology of adoptively transferred CTL by tracking the persistence and trafficking of infused clones utilizing vector-specific Q-PCR performed on peripheral blood and biopsy samples, the capacity of these patient populations to mount a cellular immune response against the expressed transgenes, and the efficacy of ganciclovir to ablate HyTK⁺ T cell clones and ameliorate significant toxicities should they be observed. Adverse events attributed to these infusions on these two trials are summarized (Table 2). To date, there have been no grade IV or grade V adverse events attributed to the use of genetically modified T cells at COH.

TABLE 2
Adverse Events with an Attribution >3 (Probable, Definite)
Associated with Infusions of Genetically Modified T Cells

| Infusion Cell Dose | Grade 1 | Grade 2 | Grade 3 |
|---|---|---|---|
| 10⁸ cells/m² | Flushing | Lymphopenia × 3 | Lymphopenia × 2 |
| total = 12 infusions | Cough × 2 | WBC × 2 | |
| | WBC | ANC × 3 | |
| | Platelet | Pruritis | |
| | | Neuropathic pain | |
| 10⁹ cells/m² | Fever × 3 | Lymphopenia | |
| total = 5 infusions | Chills × 2 | Allergic Reaction | |
| | Tachycardia | Pruritis | |
| | Cough | Vomiting | |
| | HGB | Fever | |

### Example 4

### rhIL-2 Therapy to Support Survival of Adoptively Transferred T Cells

Recombinant human IL-2 is a pleiotropic cytokine that supports the survival and proliferative expansion of antigen-activated cytolytic T cells and natural killer cells, and also for promoting their differentiated functions of cytokine secretion and cytolysis. Low doses of this cytokine induce significant immunomodulation avoiding the severe side effects associated with high-dose rhIL-2 therapy. For example, in the absence of a physiologic CD4⁺ helper-response, the in vivo persistence of adoptively transferred CD8⁺ melanoma-specific CTL may be maintained, without significant toxicity, by exogenous administration of subcutaneous rhIL-2 dosed at 5×10⁵ IU/m² twice a day (Foon et al., 1986). A FDA-authorized adoptive therapy protocol at COHNMC (IRB #99183), covered by BB-IND 9149, treated children with recurrent/refractory neuroblastoma with autologous CD8⁺ T cell clones, genetically modified to express the CE7R chimeric immunoreceptor, along with low-dose rhIL-2. The study has completed enrollment and there has been 50 doses of rhIL-2 at 5×10⁵ IU/m² given twice a day without any associated adverse events of attribution >2.

### Example 5

### Tracking Circulating Genetically Modified Cells

Q-PCR using transgene-specific primers and PCR to identify clone-specific pattern of TCR usage are used to follow the persistence of adoptively transferred T cells. Quantitative real-time PCR (Q-PCR) is presently employed in a lymphoma adoptive therapy trial to track the persistence of infused scFvFc:ζ⁺ clones in the circulation of recipients. Clonally unique TCR variable beta (TCR Vβ) gene rearrangements are commonly used to assess the clonal heterogeneity of T cells. TCR Vβ transcripts of differing CDR3 length can be readily identified by RT-PCR using a multiplex spectratyping method that detects between 8 and 10 distinct Vβ subtypes within each of the 23 TCR Vβ families (Sprent and Surh, 2003). We have adapted TCR spectratyping to screen for TCR Vβ usage in polyclonal/oligoclonal populations of antigen-specific T cells. For example, we have performed spectratyping to evaluate the relative expression of TCR Vβ usage of T cell lines stimulated in vitro with influenza matrix protein 1 (MP1). This analysis revealed a strong bias towards Vβ17 usage in MP-1 specific CTLs in HLA-A2⁺ individuals (Lawson et al., 2001; Moss et al., 1991). To perform this assay, cDNA was synthesized from T cell extracted total RNA using M-MLV reverse transcriptase and primer p(dT)12-18 (GIBCO-BRL). The multiplex PCR method amplifies 46 functional genes comparing 23 TCR Vβ families in 5 reactions where each reaction contains 4 to 7 specific primers together with a single TCR Vβ constant region primer tagged with the fluorescent FAM (6-carboxyfluorescein) dye.

### Example 6

### Detection of Anti-Transgene CTL Responses Elicited by Infusions of Genetically Modified CTLs

In order to evaluate the immunogenicity of T cells engineered to co-express chimeric immunoreceptors and drug selection genes such as neomycin phosphotransferase (Neo^{R}) and HyTK, we have developed an in vitro culture system by which anti-transgene CTL reactivity of PBMC's obtained after adoptive therapy can be compared to PBMC obtained prior to exposure to the autologous engineered cell product. Briefly, 5×10⁶ PBMC responders are co-cultured with 5×10⁵ irradiated stimulator cells with the addition to culture of 5 U/mL rhIL-2 every 48-hrs. Stimulator cells are either the clone used in the patient's adoptive therapy or autologous EBV-transformed lymphoblastoid cells (LCL's). After two 7-day stimulation cycles, responding T cells elicited in vitro are harvested and assayed in 4-hr chromium release assays against the clone used in therapy, autologous LCL, or auto-LCL-transfectants expressing the drug resistance gene. Cultures stimulated with LCL serve as a positive control that the culture system generates CTL responses to recall antigens (EBV), while cultures stimulated with the clone detect responses against the transgenes.

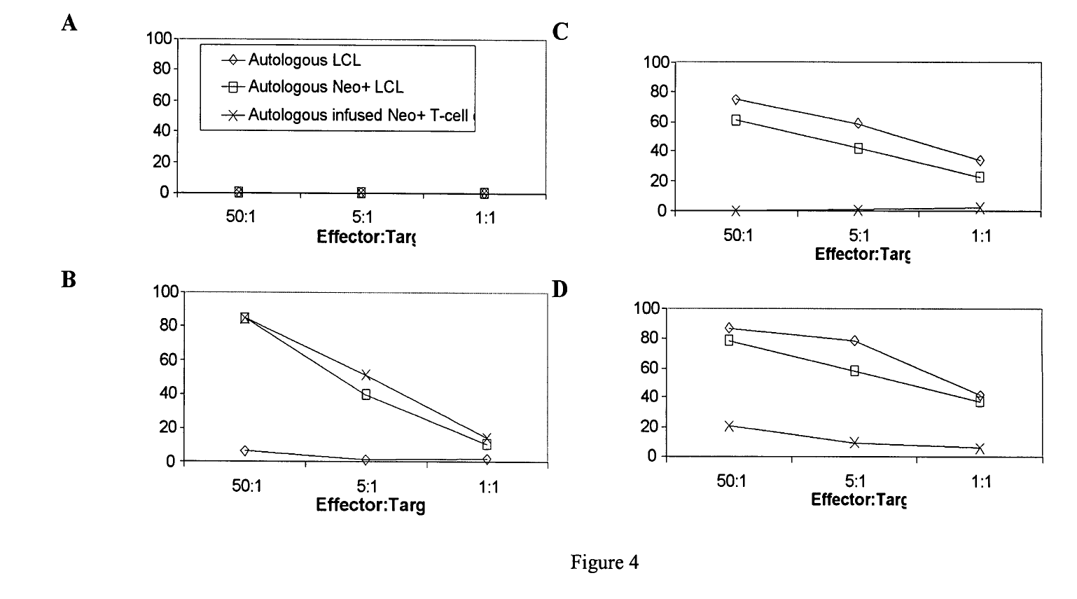

The differentiation between anti-chimeric receptor and anti-NeoR/HyTK rejection responses can be inferred by comparing clone-stimulated responders against clone (chimeric immunoreceptor⁺/selectable marker⁺) targets versus chimeric immunoreceptor⁻/selectable marker⁺ LCL-transfectants targets. This analysis was carried out on a study subject participating on COHNMC protocol IRB#98142, who after his third infusion of anti-CD20 chimeric immunoreceptor⁺/NeoR⁺ CTL-clone experienced fever and changes in peripheral blood Q-PCR signal for the clone consistent with an transgene-specific rejection response of the infused genetically-modified T cells. The results of the immunogenicity assay work-up on this patient are depicted (FIG. 4). The upper two graphs are the cytolytic activity of pre- and post-adoptive therapy PBMC responders stimulated with the T cell clone #6D10 used in therapy. We observed cytolytic activity against the clone and NeoR⁺ LCL targets from the Day⁺100 PBMC's. The cytolytic reactivity in these post-adoptive therapy PBMC responder cultures against the clone and the NeoR⁺ auto-LCL are equivalent, suggesting that the rejection response was strongly biased against NeoR and not the chimeric scFvFc:ζ chimeric immunoreceptor. This was subsequently confirmed by cloning these PBMC—all 55 analyzed clones were NeoR-specific. The lower two graphs demonstrate that EBV recall-responses could be expanded from both PBMC's responder specimens.

### Example 7

### Distribution of Genetically Modified T Cells in Lymph Node and Bone Marrow by PCR-FISH

If the transferred T cells are able to migrate to lymph nodes, the anti-lymphoma activity might be limited if the malignant cells are replicating within the lymph node architecture that is inaccessible to the genetically modified T cells. Therefore, the distributions of both the infused T cells and the lymphoma cells within the lymph node are determined. This is accomplished with PCR-ISH, a technique that can resolve T cells in the removed lymph node tissue into distinct populations based upon the presence of the introduced hygro gene, and determine the dispersal of the genetically modified T cells with respect to the distribution of the follicular lymphoma cells. A solution containing 1× Self-Seal Reagent (MJ Research Inc., South San Francisco, Calif.), 1× PCR buffer, 2.5 mM MgCl₂, 200 μM dNTPs, 50 pM of hygro-specific primers (hygroF: 5′ CGTGCACAGGGTGTCACGTTGCAAGACC 3′ (SEQ ID NO:4); hygroR: 5′ CCTCGTATTGGGAATCCCCGAACATCGC 3′ (SEQ ID NO:5)) and Taq polymerase (0.15 U/μl) is prepared. A portion (50 μL) of this PCR mixture is applied to the deparaffinized and proteinase K-treated (20 μg/mL for 20-40 min) serial histologic tissue sections of the excised lymph node and after coverslips have been applied, the slides are placed into the Slide Chambers Alpha Unit of a PTC-200 thermocycler (MJ Research, Inc, South San Francisco, Calif.). After 30 cycles of denaturation (94° C., 1 min), primer annealing (60° C., 2 min) and primer extension (72° C., 2 min), the coverslips are removed by soaking the slides in hybridization buffer (5×SSC, 50% formamide, 0.5% Tween 20). The slides are air-dried and Frame-Seal Chambers (MJ Research Inc., South San Francisco, Calif.) are placed on the slides.

The PCR product is detected by in situ hybridization using a ‘cocktail’ of three hygro-specific oligonucleotides labeled with digoxigenin-11-dUTP (DIG; Boehringer), which are in sense orientation and internal to the PCR primer binding sequences (hygli: 5′ CGA TCTTAGCCAGACGAGCG 3′ (SEQ ID NO:6); hyg2i: 5′ CTGGCAAACTGTGATGGACG 3′ (SEQ ID NO:7); and hyg3i: 5′ CCTCGTGCACGCGGATTTCG 3 (SEQ ID NO:8)). The oligonucleotide probes (30 ng in 5×SSC, 50% formamide, 0.5% Tween-20, 100 μg/ml sonicated salmon sperm DNA and 5× Denhardt's solution) are hybridized to tissues overnight at 42° C. in the Slide Chambers Alpha Unit of the thermocycler. The slides are then washed in 2×SSC, 0.5% Tween-20 for 30 min at 42° C. followed by 0.2×SSC, 0.5% Tween-20 for 20 min at 25° C. Hybridized probes are detected with AP-conjugated anti-DIG mAb (150 mU/mL) and nitroblue tetrazolium-5-bromo-4-chloro-3-indolylphosphate toluidinium substrate. The presence of hygro DNA is indicated by a purple, cell-associated precipitate, and can be visualized by incident light microscopy (data not shown). Positive controls consist of CD19R+HyTK⁺ T cells immobilized in paraffin wax. Negative controls for PCR include tissue processed without Taq polymerase or without hygro-specific primer pairs and with irrelevant oligonucleotide probes specific for the neo gene. Identical procedures performed on lymph nodes from individuals who have not received genetically modified T cells also serve as a negative control.

### Example 8

### Sleeping Beauty Transposon System

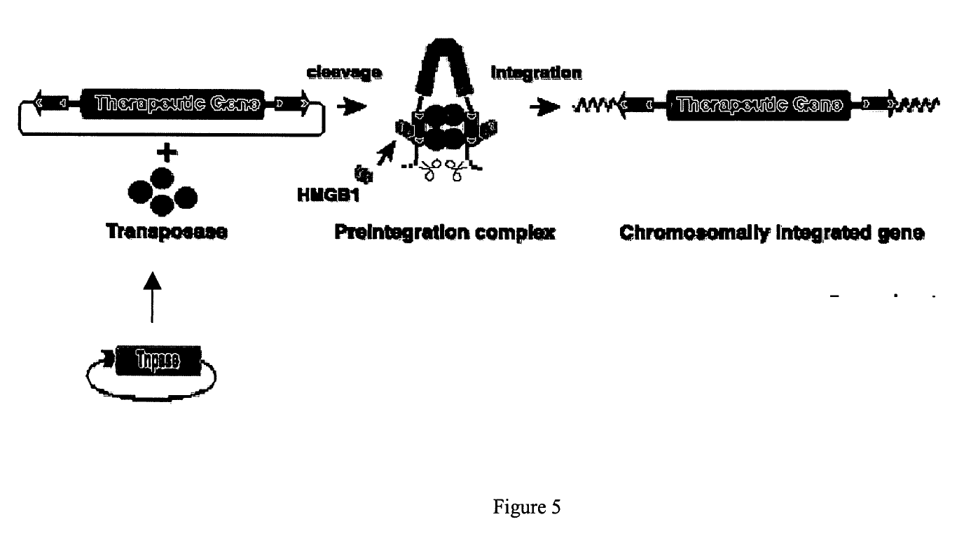

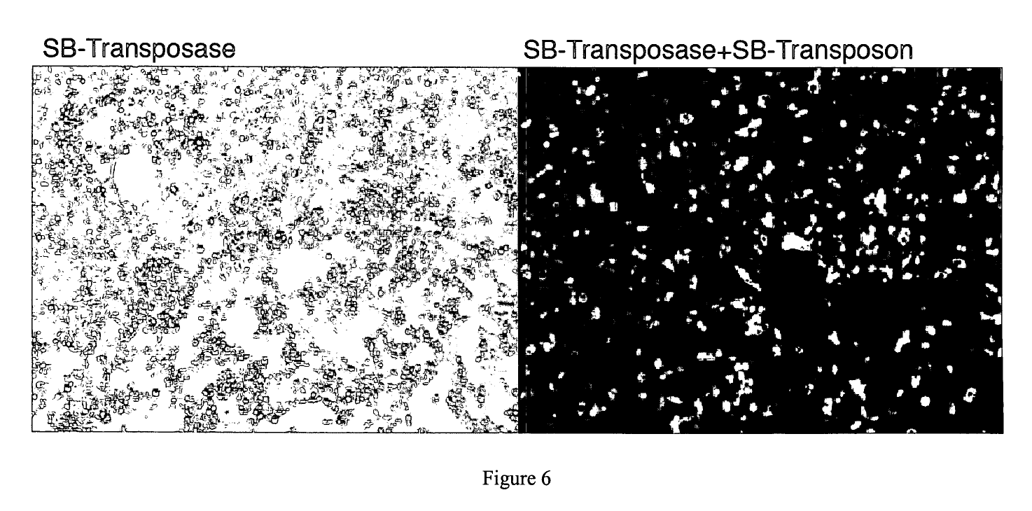

The sleeping beauty transposon system (SB) is a molecular reconstruction of a transposon taken from a Tc1/mariner-type found in the fish genome, which no longer acts as a transposon in fish (Plasterk et al., 1999). The SB system is a two plasmid transfection system with one plasmid expressing the SB-transposase and other plasmid containing the SB-transposon with the gene of interest inserted between two inverted repeats (FIG. 5). To determine the efficacy of SB based cell transduction we transfected 293FT cells with 0.5μg of the SB-transposase and 0.5 μg of the SB-transposon containing EGFP expressed from the CMV promoter (FIG. 6). EGFP expression was characterized by microscopy at 48-hours post-transfection and compared to SB-transposase transfected controls. Varying the SB transposase amino acid concentration in the N-terminal binding domain has been shown to increase transduction efficiency of larger SB transposons (Yant et al., 2004). To test the effectiveness of the newly constructed SB transposase we co-transfected 293FT cells with either the wildtype SB transposase (pCSB11, 100 ng) or the new improved SB transposase (pHSB2, 100 ng) and the transposon (pT2/BHEGFP, 500 ng). The improved pHSB2 transposase increased overall transduction efficiency as detected at day 50 post-transfection (Table 3).

TABLE 3
Increased long-term gene transfer using SB system

| | | GFP+ |
|---|---|---|
| Transposase | Transposon | % after 50 days |
| None | pT2/BHEGFP | <1% |
| pCSB11 (wildtype) | pT2/BHEGFP | 20% |
| pHSB2 (Modified) | pT2/BHEGFP | 31% |

### Example 9

### RNA Interference and HLA Knockdown (shRNA vs. siRNAs)

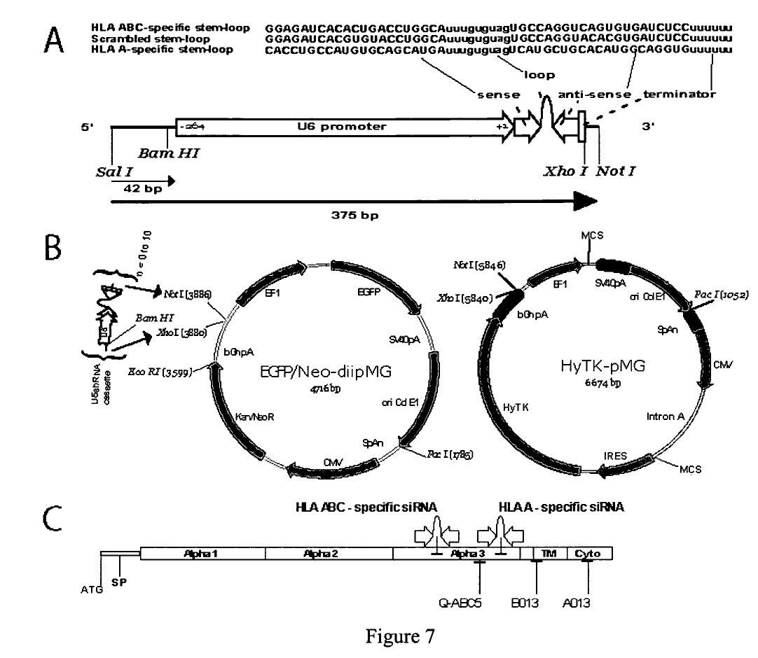

Two forms of RNA mediated gene silencing are now being used for targeted gene knockdown, shRNA (mRNA targeted) and siRNA (promoter targeted). The preliminary studies shown below are weighted towards (shRNAs) while the siRNA data was recently published (Morris et al., 2004). SiRNAs are small nucleic acid reagents that, in contrast to virally-derived proteins, are unlikely to elicit an immune response. Therefore, we developed a strategy to express intracellular siRNAs, homologous to a sequence conserved in most classical polymorphic HLA-A, -B and -C loci, as hairpin transcripts from mammalian RNA polymerase III (Pol III) promoters (Lee et al., 2002; Brummelkamp et al., 2002) to achieve suppression of major histocompatibility complex (HLA) class I cell-surface expression. Given that the design of HLA ABC-specific siRNA is constrained by choosing 21-base pair (bp) binding-sites homologous to the majority of classical class I alleles, which may include sites associated with adverse siRNA position effects, and since multiple endogenous genes need to be simultaneously targeted to achieve down regulation of HLA molecules, we developed a system to titrate/augment expression of shRNA using a plasmid vector by increasing the number of U6 promoters and shRNA cassettes (FIGS. 7A and 7B). For further details of this plasmid vector, see U.S. patent application Ser. No. 11/040,098 filed on 24 Jan. 2005 and international application No. PCT/US2005/002172 filed on 24 Jan. 2005, each incorporated herein by reference. See also, Gonzalez et al. (2005). FIG. 7C depicts an HLA class I molecule and the relative position of the siRNA binding sites used.

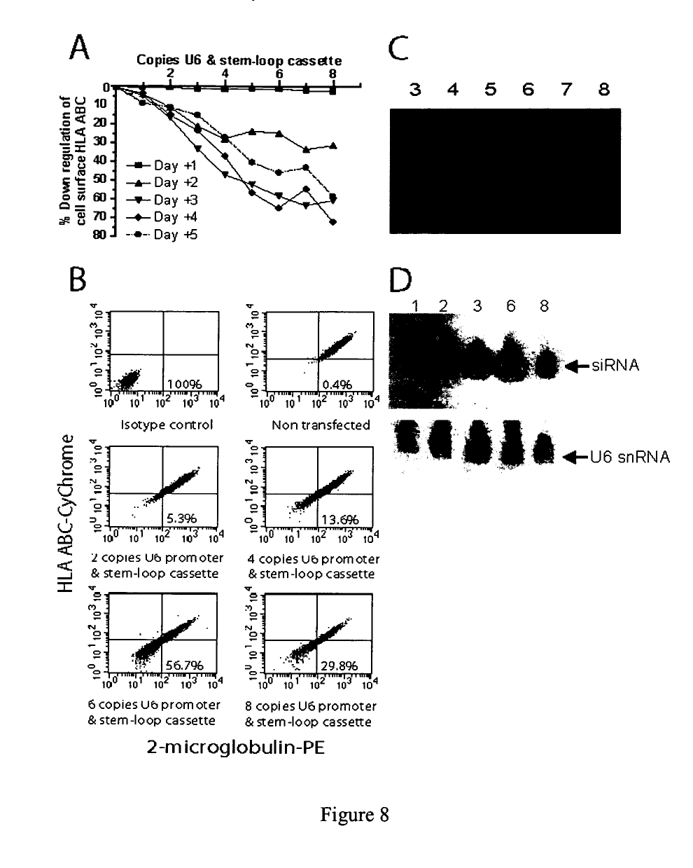

To titrate/augment RNAi-effects, we transiently down regulated HLA class I expression on Jurkat cells, a T cell line expressing HLA A*0301/0301 B*0702/3503 Cw*401/0702, transfected with a panel of DNA vectors containing between 0 and 8 copies of the U6shRNA cassette. A flow cytometry kinetic study demonstrated that the down-regulation of HLA ABC antigens peaked between three to four days after transfection (FIG. 8A), reflecting the time required to achieve sufficient shRNA expression and RNAi to prevent replacement of HLA A, HLA B and HLA C molecules on the cell-surface. Strikingly, increasing the copy-number of the U6shRNA cassettes from 1 to 8 resulted in a steady increase in RNAi, with a maximal 19-fold improvement in the siRNA-effect. Down-regulation of HLA ABC expression was specific as cells transfected with a DNA plasmid expressing a scrambled version of the HLA ABC-specific shRNA showed negligible loss of HLA class I cell-surface expression. We were also able to achieve durable down regulation of HLA ABC levels as a result of augmented shRNA expression. While expression of two copies of the U6shRNA cassettes resulted in 5.3% of the G418-resistant T cells with down-regulated protein expression of both HLA ABC and β2-microglobulin (β2-m), this percentage increased approximately 11-fold when 6 copies of the U6shRNA cassettes were expressed (FIG. 8B). Southern blotting analyses confirmed that the G418-resistant Jurkat cells had integrated the correct number of U6shRNA cassettes (FIG. 8C). The siRNA-mediated down-regulation of HLA ABC has been maintained for an extended period of time, as transfected Jurkat cells continue to demonstrate down-regulation of HLA ABC protein expression after 6 months of passage in tissue culture. No β-IFN production, a non-specific effect induced by expression of shRNA, was detectable in the cells expressing multiple copies of the U6shRNA cassettes.

The degree of HLA ABC protein down-regulation correlated with the level of expression of stem-loop dsRNA as confirmed by Northern analyses of the shRNA constructs (FIG. 8D). The ability to down-regulate HLA ABC protein expression peaked with the introduction of 6 copies of the shRNA cassettes in stable transfectants (FIG. 8B), while 7 to 10 copies of the shRNA cassettes showed a slight decrease in HLA down-regulation, which was consistent with a relative decline in their intracellular RNA expression (FIG. 8D). The reason(s) for this loss in efficacy and expression with greater than 6 copies of the cassette are not clear, but could include local chromatin alterations resulting in relative loss of Pol III expression or a selective disadvantage of stable over-expression of anti-HLA ABC shRNA.

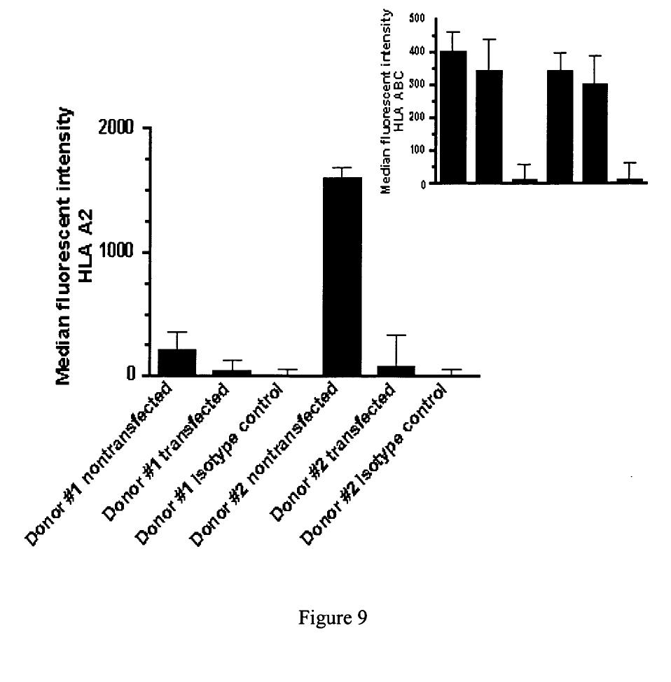

To demonstrate the activity of shRNA in primary T cells and avoid auto-deletion of T cells that had lost expression of classical HLA class I molecules by autologous NK-T cells present in PBMC, a new shRNA was constructed with a 21 nucleotide sequence completely homologous to most HLA A alleles, but which contained bp mismatches with HLA B and C alleles. To generate HLA A2^{neg} T cells that could be eliminated in vivo by ganciclovir-mediated ablation, heterozygous and homozygous HLA A2⁺ primary T cells were transfected with the HyTK-pMG plasmid, modified to express 6 copies of the HLA A-specific shRNA (FIG. 7A). Hygromycin-resistant T cells could be demonstrated to have down-regulated HLA A2-expression, relative to drug-resistant parental T cell controls that do not express the shRNA (FIG. 8B). As expected, there was only a small decrease in the binding of the niAb specific for HLA ABC to the T cells that had down-regulated HLA A2 expression, reflecting the fact that this mAb clone recognized an epitope also present on HLA B and C molecules (FIG. 9 insert). To our knowledge, this is the first demonstration of siRNA-effects in primary T cells electroporated with a DNA plasmid, a vector system that is currently being evaluated in adoptive immunotherapy clinical trials. The ability to disrupt antigen presentation by down-regulating HLA gene expression using RNAi is an approach to avoiding T cell-mediated immune recognition, which might be used to facilitate transplantation and/or adoptive immunotherapy between HLA-divergent individuals or to prolong the in vivo survival of transferred T cells that express vector-encoded immunogenic transgenes and is a step toward the construction of pre- prepared “universal” T cells expressing tumor-specific chimeric immunoreceptors, that dock with antigen independent of HLA, which could be readily available for adoptive immunotherapy of HLA-disparate recipients.

### Example 10

### Methodology: Sleeping Beauty Gene Transfer System

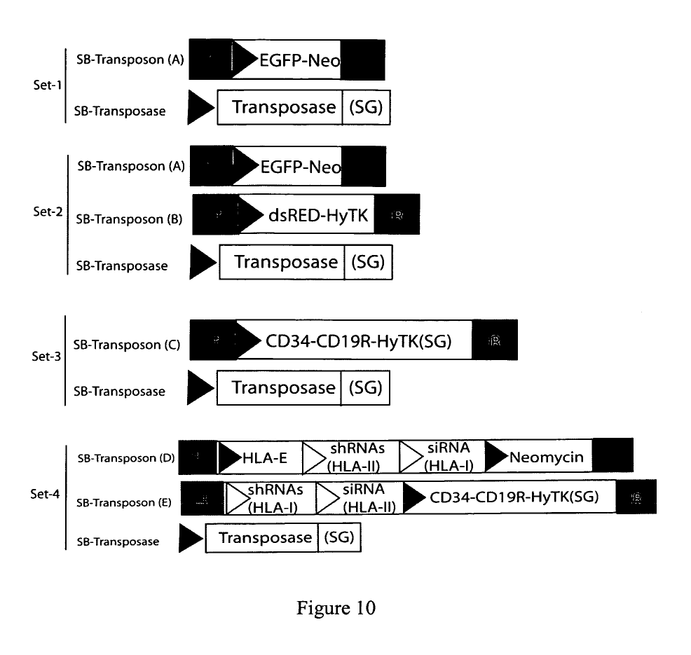

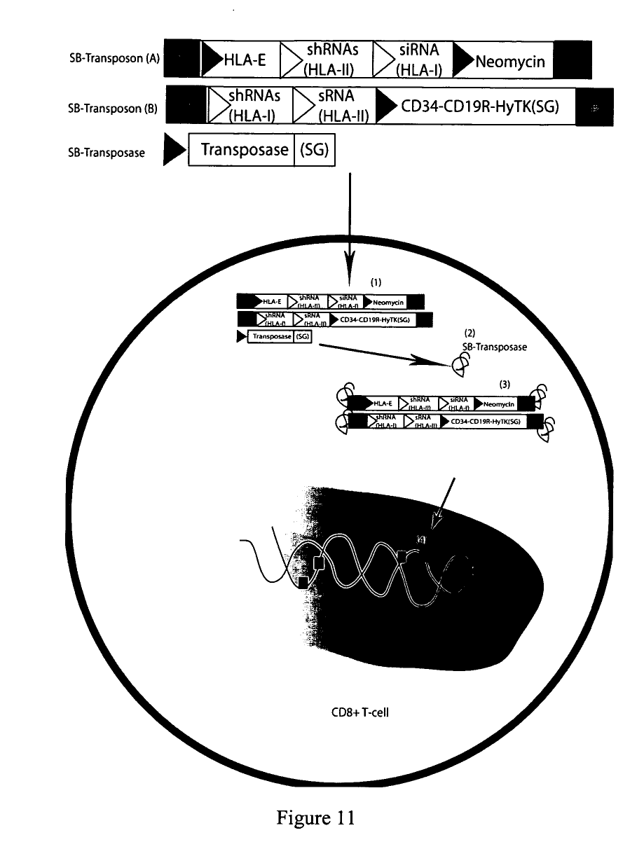

At COH, non-viral gene transfer has been used to introduce desired transgenes in T cells used for four clinical trials. Compared with using retroviral transduction to generate T cells for therapy, our electroporation approach is less expensive and has not been associated with T cell leukemia's resulting from integration of viral promoter next to an oncogene. However, efficiency of non-viral gene transfer is low (˜1%). To improve the transfection efficiency, the sleeping beauty (SB) transposon system (FIG. 5) is used with slight modifications (FIGS. 10 and 11) (Liu et al., 2004; Geurts et al., 2003; lzsvak et al., 2000). Initial experiments determine the relative transfection efficiency of the SB system. Preliminary data using a EGFP reporter gene in a DNA plasmid, has demonstrated that the Amaxa Nucleofector™ system, which is capable of gene transfer into non-proliferating T cells, results in increased number of transiently-transfected T cells (50%), compared with primary T cells electroporated with the Eppendorf Multiporator device. However, the incidence of stably transfected (drug-resistant) T cells using either electroporator system remains about the same, ˜1%.

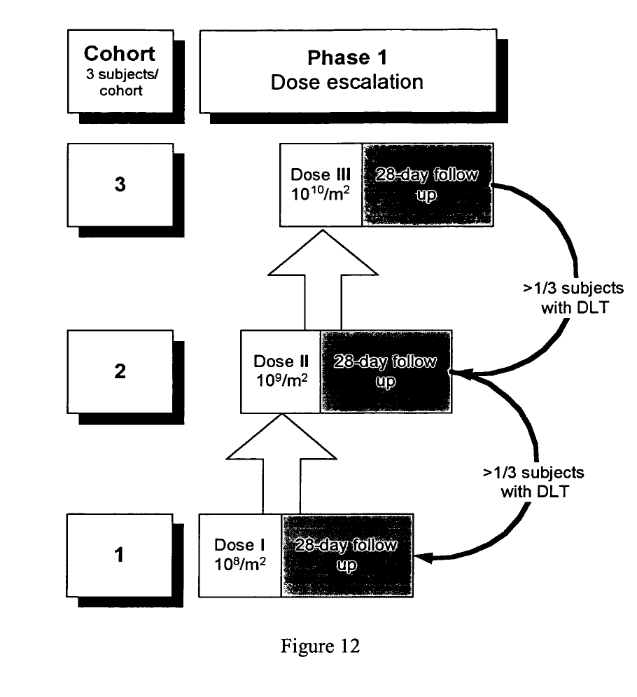

To evaluate transfection efficiency of SB system in T cells the SB-transposase is co-transfected with an SB transposon containing the EGFP-Neomycin reporter/selection fusion gene expressed from a CMV Pol-II promoter (FIG. 10, Set 1). The SB system is compared with the EGFP expressed from the pMG plasmid backbone, which is currently in clinical trials (CD19R/HyTK-pMG, FIG. 12). The SB transposon and transposase has already been obtained and shown to be operable in transfected 293FT cells (a gift from M. Kay, FIG. 6 and Table 3). To determine optimal transfection efficiency varying ratios of SB-Transposon (A) to SB-Transposase totaling of 7.5 μg (1:1, 1:5, 5:1 (A) to SB-Transposase respectively, FIG. 10, Set 1) are transfected in 8×10⁶ T cells using the Amaxa Biosystem Nucleofector™ as well as the control pMG-EGFP plasmid as described above. Gene transfer is validated by FACS for expression of EGFP at day 2 post-transfection and efficiency of integration is determined by plating at 0.3 T cells/well stimulated to grow in situ with a cocktail containing 30 ng/ml of OKT3, 50 units/ml IL-2 and double cell irradiated feeder layer of 10⁵ PBMC/well and 2.0×10⁴ LCL/well and limiting dilution 96 well cloning plates in cytocidel concentrations of G418. The number of EGFP⁺ wells over the total wells plated give a measure of integration efficiency.

A critical parameter to using modified T cells for clinical trials is that the modified cells contain only one integrated copy of the transgene. The sleeping beauty system has the potential for high-efficiency integration, up to ˜10% in glioblastoma cells following electroporation (Ohlfest et al., 2004). Consequently, to evaluate the number of integrants/cell an ALU-based PCR is used (Morris et al., 2004). In the event multiple integrants are seen and to rule out that greater than one population of transfected T cells was generated a Southern analysis is undertaken to validate a single band using a specific probe to the neomycin gene. With the increase in transfection efficiency, (1) multiple SB transposon plasmids are used to introduce single copy of multiple genes, and optionally (2) a non-immunogenic selection approach to generating CD19 specific T cells is developed for use in clinical trials. To determine the feasibility of using greater than one SB transposon (1), varying combinations and ratios of a SB-Transposon (A) containing EGFP-Neo and SB-Transposon (B) containing Hygro-dsRED2 and the SB transposase (FIG. 10, Set 2) are co-transfected as described previously with ratios (1:1:1, 1:1:5, and 5:5:1, plasmid A:B:SB-Transposase, respectively).

The development of a non-immunogenic selection method (2) is optional for the present invention. This selection method is capable of producing T cells for adoptive transfer that can avoid immune mediated clearance by the recipient. A chimeric immunoreceptor is generated which fuses a non-immunogenic selectable epitope to the CD19 chimeric immunoreceptor. Either CD19R or this chimeric immunoreceptor is used in accordance with the present invention. The chimeric immunoreceptor, e.g., CD19R, is cloned into the SB system (FIG. 10, Set 3) and the optimal conditions for transfection determined as described previously. Two days post-electroporation T cells with cell surface expression of CD19R are isolated and rapidly expanded with OKT3 and IL-2. At the end of the second 14-day growth cycle (mediated by OKT3) the cell surface expression of the CD19R is assessed using anti-CD19 and anti-FC. Preliminary data has demonstrated an inability to expand CD19 specific T cells in the absence of drug selection and absence of the SB system using the Amaxa electroporator. Functional activity of these T cells is evaluated below.

### Example 11

### Methodology: siRNA Design to Silence Classical HLA-I and II T Cell Expression

We have constructed and characterized shRNAs targeting a conserved region of the classical HLA-I mRNA to generate T cells that can avoid recognition by CD8⁺ T cells. To generate genetically modified T cells that can avoid recognition by recipient HLA-I disparate CD8⁺ T cells and HLA-II-disparate CD4⁺ T cells, HLA-I and HLA-II cell-surface expression are suppressed by using shRNAs acting at the level of mRNA, to target conserved nucleotides in the HLA-I heavy chain and 2 HLA-II heavy chains in a fashion similar to that described above with respect to HLA-I). The shRNAs are expressed under pol III (U6) promoters as multiple cassettes to maximize down-regulation. To achieve an increased RNAi-mediated effect on both HLA-I and II expression, siRNAs acting on the HLA-I and HLA-II promoters are also used. Recently, it has been demonstrated that siRNA directed to a genes promoter as opposed to mRNA can suppress gene expression by transcriptional gene silencing (TGS) (Morris et al., 2004; Kawasaki and Taira, 2004). A minimum of 4 sites in each the classical HLA-I and II promoters are selected and siRNAs constructed (previous work has shown ˜1 in 3 siRNAs are effective at TGS). The 4 candidate siRNA target sites are designed to specifically target CG rich regions and the TATA box, previously shown to be effective (Morris et al., 2004). The U6-expressed shRNAs, as multiple cassettes (n=1 to 6) and siRNAs are developed from Ambion Silencerm or directly from PCR products (Castanotto et al., 2002). The respective RNAs are screened initially by transient transfection in Jurkat T cells (Amaxa Nucleofector™, putatively nuclear specific) and relative HLA-I and II expression are determined by real-time kinetic RT PCR and flow cytometry at 24, 48, 72, 96 and 168 post-siRNA transfection. The most potent shRNA and siRNAs are selected and expression cassettes generated and cloned into the developing sleeping beauty transposon plasmid system (FIGS. 10 and 11).

To reduce the likelihood of promoter interference in the therapeutic SB system, a 3-plasmid co-transfection scheme is used (FIG. 11). The three-plasmid SB system consists of the SB-Transposase fused to thymidine kinase suicide gene (a gift from P. Hackett), the SB-Transposon (D) expressing chimeric HLA-E (to avoid NK mediated targeting), anti-HLA-II-shRNAs (mRNA targeted), anti-HLA-I-siRNA (promoter targeted siRNA), and the Neomycin phosphotransferase selection gene and the SB-Transposon (E) expressing the anti-HLA-I-shRNAs (mRNA targeted), anti-HLA-II (promoter targeted siRNAs), CD19R fused to the HyTK suicide/selection gene (FIGS. 10, Set 4 and FIG. 11) (Cooper et al., 2003; Cooper et al., 2004).

Since classical HLA molecules are not expressed after RNAi-mediated suppression, a chimeric HLA-E, kindly provided by Dan Gerharty, which uses the HLA-A2 signal sequence to achieve surface expression (Lee et al., 1998) is employed. Due to the fact that the added HLA-E can also be targeted by the shRNAs directed towards classical HLA-I, conservative point-mutations that do not affect the coding capacity of HLA-E, but elude HLA-E mRNA degradation by shRNA targeting are introduced. Furthermore, a FLAG-tag is expressed at the amino terminus of the chimeric HLA-E, and mAb specific for FLAG epitope is used to distinguish endogenous HLA-E from introduced chimeric HLA-E. The chimeric HLA-E is expressed under strong Pol-II viral promoter, and therefore avoids down regulation by targeting endogenous promoter driving HLA E expression.

All three plasmids are constructed and transfected using the Amaxa Nucleofector™ at varying ratios (5:5:1, 1:1:1, and 1:1:5, SB-(D):SB-(E):SB-Transposase, respectively) into primary T cells obtained from a HLA A2⁺ DRB1*0401⁺ influenza-seropositive healthy donor (FIG. 11). The cells are numerically expanded in cytocidal concentrations of hygromycin B and G418 using repetitive 14-day OKT3-mdiated growth cycles. The SB-transfected T cells are screened for cell surface expression by flow cytometry using mAb specific for HLA ABC, HLA-DR, and Flag. The shRNA or siRNA expression is determined by Northern Blot analysis using the respective siRNA or shRNAs antisense strand as a probe. Each of these transfections is repeated 5 times to achieve statistical significance. Safety of genetically modified T cells is enhanced if there is only one integrated copy of the inserted genetic material. Indeed, this is currently a release criteria for manufactured T cell clones. To determine the number of integrated SB-Transposons relative copies of integrated SB determined by an ALU based semi-quantitative PCR (Morris et al., 2004) is used.

While, flow cytometry can establish phenotype, 4-hour chromium release assays (CRAs) and 48-hour cytokine production are used to establish that the genetically modified T cells are functionally resistant to T cell recognition and NK-mediated lysis. The HLA A2⁺ DRBI⁺ HLA^{null} T cells are used as targets/stimulators by incubating with 1 μg/mL HLA A2-restricted peptide (GILGFVFTL (SEQ ID NO:9)) or HLA-DR-restricted peptide (FVFTLTVPSER (SEQ ID NO:10)) derived from influenza matrix protein 1 (MP1). The same T cells that are not incubated with peptide serve as specificity controls. Autologous T cells are isolated by flow cytometry-sorting using MP1-specific HLA A2-tetramer (purchased from Beckman Coulter) to obtain CD8⁺ MP1-specific T cells and MP1-specific HLA DRBl-tetramer (purchased from Beckman Coulter) to obtain CD4⁺ MP1-specific T cells effector/responder T cells. If necessary to obtain sufficient numbers of MP1-specific T cells, the sorted cells are numerically-expanded using OKT3. The tetramer+effector/responder T cells are incubated with the genetically modified ⁵¹Cr-labeled target/stimulator T cells and chromium release and γ-IFN cytokine production. Absence of specific chromium release or γ-IFN production is consistent with loss of functional expression of HLA-I/II. To validate that the genetically modified HLA^{null} T cells are resistant to NK-mediated lysis, the T cells are loaded with ⁵¹Cr and used as targets by the NK-T cell line NK-92 (obtained from DSMZ -German Collection of Microorganisms and Cell Cultures). HLA-I^{neg}K562 cells are a positive control for this CRA.

### Example 12

### Methodology: In vitro Activity of CD19R⁺ T Cells Genetically Modified with Sleeping Beauty

An influenza matrix protein 1 (MP1)-specific CD8⁺ T cell clone, obtained by flow sorting MP1-tetramer⁺ T cells from a HLA A*0201 healthy donor, are activated on day 0 with OKT3 (anti-CD3) and genetically modified using the optimal SB plasmid ratio defined in Aim 1. The panel of SB-(D), SB-(E), and SB-Transposase plasmids (FIG. 10, Set 4) are used to introduce siRNA targeting HLA promoters and classical HLA-I and HLA-II, enforce expression of chimeric HLA-E, and introduce the CD19-specific chimeric immunoreceptor (CD19R) and the HyTK selection/suicide or Neomycin selection genes. A new multi-function molecule has been generated, which combines the chimeric immunoreceptor, CD19R, fused in frame with HyTK. This CD19R-HyTK has been successfully expressed on the surface of hygromycin-resistant T cells, which have redirected specificity for CD19 antigen. A similar approach is used to generate the CD19R-HyTK receptor (described in FIGS. 10 and 11), so that hygromycin-resistant T cells express the chimeric immunoreceptor. The transfected T cells are numerically expanded in the presence of cytocidal concentrations of hygromycin B and G418 and evaluated by flow cytometry for loss of cell-surface expression of HLA ABC and HLA DR. To correlate loss of HLA expression with RNA-mediated effect, siRNA and shRNA expression are determined by Northern blot analysis. Untransfected MP1-specific T cells serve as control. The genetically modified T cells are assessed by flow cytometry for expression of CD19R (using anti-Fc) and HLA-E (using anti-FLAG). The overall integration frequency is determined by semi-quantitative PCR using SB transposon-specific and ALU-based primers (Butler et al., 2001) as described above. To determine the extent to which Dicer may become saturated, as multiple shRNA cassettes targeting the same mRNA are expressed in each cell, (1) measure Dicer expression is measured by real-time RT PCR, and (2) titrate Dicer activity in the selected SB transfected cells and non-SB transfected T cells is titrated by transfecting synthesized siRNAs at (0.1, 1, 10, 50, 100 and 500 nM) targeting the HLA-E.

The ability of genetically modified HLAnull CD19R⁺ HLA-E⁺ to be activated by both MP1 and CD19 antigens is accomplished using a panel of HLA A2⁺ target cells that have been genetically modified to express truncated CD19 (tCD19) or a fusion protein of hygromycin and MP1 (HyMP1), to express full-length MP1 in hygromycin-resistant cells. These cells are loaded with ⁵¹Cr and used as targets for the genetically modified T cells in a 4-hour chromium release assay (FIG. 3). In addition to activation for cytolysis, the genetically modified T cells are evaluated for their ability to be produce IFN-Y in response to CD19 and MP1 presented by the panel of CD19⁺ and/or MPl+stimulator cells. These data validate that the introduced CD19-specific chimeric immunoreceptor (and endogenous MP1-specific TCR, serving as a positive control) continues to function in genetically modified T cells that have lost HLA expression.

### Example 13

### In vivo Anti-Tumor Activity of Genetically Modified CD19-Specific T Cells

The T cell clones generated using the SB system are used in an in vivo model. A NOD/scid mouse model of CD19⁺ malignancy has been established and non-invasive biophotonic imaging has been used to quantify the size of tumor expressing Firefly luciferase (ffLuc). Bioluminescent imaging after infusing D-luciferin measures the amount of tumor before adoptive immunotherapy. The ability of the genetically modified CD19-specific T cells to eradicate the subcutaneously deposited established tumor cells is investigated as shown (Table 4). Control mice with tumor do not receive adoptive immunotherapy or are intravenously infused with universal genetically modified T cells (mouse groups C and E) along with ganciclovir to mediate ablation of the T cells expressing the TK gene (groups B and E). T cells genetically modified to express CD19R (using the plasmid CD19R/HyTK-pMG, described in FIG. 1), but not RNA, serve as a positive control (group D). Preliminary data demonstrates that adoptive transfer of 2.0×10⁷ CD19-specific T cells can eradicate controlled tumor in this mouse model. Biostatistical modeling indicates that mice in groups of 10 are sufficient to evaluate for statistical differences between the treatment groups (Table 4). Preliminary data has shown that there is no difference between groups A and B. It is also expected that there is no difference between groups C and D, but that there are significance differences between groups A/B and C and/or D, which is diminished compared with group E.

TABLE 4
Experimental Groups to Evaluate in vivo Efficacy of CD19-Specific Universal T Cells

| Group (10 | CD19⁺ffLuc⁺ | | | IL-2 (25,000 | Imaging after |
|---|---|---|---|---|---|
| mice/group) | Daudi | T cells | Ganciclovir | units/injection) | D-luciferin |
| A | 10⁶ s.c. day 0 | None | No | Mon-Wed-Fri | Days 3, 6, 9, 13, |
| | | | | | 26, 20, 22, 29, |
| | | | | | 35+ |
| B | 10⁶ s.c. day 0 | None | Yes | Mon-Wed-Fri | Days 3, 6, 9, 13, |
| | | | | | 16, 20, 22, 29, |
| | | | | | 35+ |
| C | 10⁶ s.c. day 0 | CD19-specific | No | Mon-Wed-Fri | Days 3, 6, 9, 13, |
| | | universal T cells | | | 16, 20, 22, 29, |
| | | 20 × 10⁶ i.v. on | | | 35+ |
| | | days 7, 14, 21 | | | |
| D | 10⁶ s.c. day 0 | CD19-specific T | No | Mon-Wed-Fri | Days 3, 6, 9, 13, |
| | | cells 20 × 106 iv. | | | 16, 20, 22, 29, |
| | | on days 7, 14, 21 | | | 35+ |
| E | 10⁶ s.c. day 0 | CD19-specific | Yes | Mon-Wed-Fri | Days 3, 6, 9, 13, |
| | | universal T cells | | | 16, 20, 22, 29, |
| | | 20 × 10⁶ i.v. on | | | 35+ |
| | | days 7, 14, 21 | | | |

### Example 14

### Pilot/Phase I Trial

In general, patients with induction-failure or in second or higher relapse B-ALL, have a poor overall survival (FIG. 11). These patients are candidates for evaluating the efficacy of novel therapies. In the clinical trial, the capacity of universal genetically modified CD19R⁺ T cells to mediate an anti-leukemia-effect in patients with high risk B-lineage ALL is studied. This pilot clinical trial has been designed such that CD19-specific universal T cells are pre-prepared and immediately available for infusion. T cell dose escalation is structured in cohorts of 3 subjects. Subjects are eligible to receive low-dose rhIL-2 to support in vivo T cell persistence.

Patient Population

9 research participants of any age with CD19⁺ ALL that is resistant to induction therapy or is in ≧2^{nd} relapse are enrolled in the trial. Groups of 3 subjects/cohort are assigned to receive the universal CD19-specific T cell clone, beginning at dose-level I (10⁸/m²), followed by dose-level II (10⁹/m²), culminating with dose-level III (10¹⁰/m²). The rules for dose-escalation and de-escalation are described below.

Production of Clinical Grade Plasmid DNA Vector

The plasmid DNA vector to express CD19-specific immunoreceptor, HyTK selection/suicide gene, siRNA to down regulate classical HLA-I and HLA-II, and enforced expression of HLA E, used for genetic modification of umbilical cord blood T cells is produced in the CBG. Production of plasmid CD19R/HyTK-pMG, which co-expresses the CD19R and HyTK genes for use in a clinical trial, is currently supported by the National Gene Vector Laboratory (NGVL) and is being used in IND BB-11411 for adoptive immunotherapy of follicular lymphoma. All plasmid DNA manufactured in the CBG is produced and linearized according to the CBG's FDA Drug Master File (DMF BB-MF 9778). All aspects of DNA production are according to SOP beginning with the creation of a Bacterial Master Cell Bank (BMCB).

Preparation of Universal CD19-Specific T Cell Clone Derived from Umbilical Cord Blood

Manufacturing of the universal T cell product occurs prior to subject enrollment in the Pilot/Phase I trial and is outlined (FIG. 2). After obtaining informed consent, umbilical cord blood (150 to 200 mL) is collected from the placenta and cord of healthy neonates after clamping/cutting the umbilical cord (IRB #03076). The product is transferred to the CBG Quality Assurance (QA) department for logging and tracking and then released to manufacturing for initial processing. A sample of is archived in liquid nitrogen by the QA Department of the CBG for retrospective analysis, if required as mandated by our Quality Systems Policies and Procedures. Another sample is tested for sterility, viability and mycoplasma contamination (as described in Table 1 Test Panel A and FIG. 2). A dedicated team carries out manufacturing, with release testing performed by individuals in the Quality Control (QC) Department dedicated to this project. Records are handled by the QA Department who are also responsible for the final release of all biologic materials. The manufacture of the CD19-specific UCBT is based on methods described above. After cloning and at the end of recursive 14-day expansion cycles, a bank of 10¹¹ T cells are cryopreserved, which is sufficient to infuise the 9 subjects in this trial (assuming maximum BSA of 2 m²/patient). Aliquots of T cells from this bank undergo release testing (Test Panel C in Table 1 and FIG. 2), and upon passing, a certificate of analysis is completed documenting that the bank is ready for infusion.

Quality Control/Assurance Procedures for DNA and T Cell Production/Release

Manufacturing in the CBG is performed according to Standard Operating Procedures (SOP's) created by the process development staff and reviewed by the Principal Investigator, Manufacturing Supervisors and the Quality Assurance (QA) and Quality Control (QC) Departments. Documents are controlled by the QA Department including revision, distribution, collection of obsolete versions, batch record issuance, collection, and archiving. All personnel who execute protocols are trained on the protocols prior to execution and receive hands on training by qualified individuals. Records of training are kept on file by the QA Department. All raw materials used in the manufacturing in the CBG are of suitable quality for use in clinical studies. Cryopreserved product intermediates and final products are controlled by the QA Department. Batch records are produced to document all procedures and materials used in the manufacturing and testing of biologics in the CBG. Batch records (including labels to be used during processing to identify samples and reagents) are issued by the QA Department, completed by the manufacturing or QC staff (for production and testing respectively), and returned to Quality Assurance for review and archiving. This includes all calculations and measurements made during production and the identity of all patient products. Final release of DNA and cell products from the CBG occurs following review by the QA and CBG management team to ensure that all required testing has been performed and that all specifications have been met. The QA Department has the final authority to approve or reject all drug products produced in the CBG.

Evaluation of Adoptive Immunotherapy Procedure

Long-term follow-up of research subjects who have received genetically modified T cell products is conducted in accordance with recent advice and recommendations provided to the FDA by the Biological Response Modifiers Advisory Committee. This program fulfills all the responsibilities as outlined in 21 CFR 312 subpart D. The monitoring/auditing plan is carried out per the Phase I Category 2 algorithm detailed in the Data and Safety Monitoring Plan in the “Human Subject” section. This monitoring plan includes a Phase I tracking log that contains data on every research participant and is reviewed by the Data and Safety Monitoring Board (DSMB) monthly. Protocol Adherence Evaluations are conducted at least every 6 months while the research participants are in active treatment then yearly on long term follow-up (LTFU) protocol IRB #02025 while subjects are in protocol-specified post-therapy monitoring. Patients on this trial are followed indefinitely by the Program's LTFU Core.

Supportive Care Measures

Serious adverse events have not been observed in research participants receiving CMV-specific and HIV-specific CTL-clones expanded by the methods outlined above (Brodie st al., 1998; Riddell and Greenberg, 1990). In addition, the infusions of genetically modified T cells at COH have been generally well tolerated (Table 2). Nevertheless, there are several complications that might acutely occur in the research participants with the infusion of CD19-specific T cells. These include the synchronous activation of large numbers of transferred CD19-specific T cells upon recognition of CD19⁺ B-cells/B-cell progenitors resulting in pro-inflammatory cytokine release that could mediate cardiovascular changes including hypotension and vascular leak syndrome, as well as, aggregation of circulating activated T cell blasts in the pulmonary vasculature. These concerns have been addressed in this study by infusing the T cell dose in two parts and by hospitalizing research participants at the time of the T cell infusion in order to facilitate the close monitoring of the recipient. A complete history and physical exam, liver function tests, serum chemistry, and CBC are performed every 7 days for 100 days following an infusion to detect toxicities possibly attributed to the T cell infusion.

As there is a possibility that the universal genetically modified UCBT may recognize host antigens, an acute GVHD scale is used to assess and grade potential skin, liver and gut toxicities. Biopsies of tissues are performed if indicated to establish the diagnosis of allo-toxicity. The initial management for mild GVHD-type symptoms attributed to the adoptive T cell transfer is observation and follow-up. If ≧grade 2 GVHD develops after any infusion and that does not decrease in severity in response to methylprednisolone, a 14-day course of ganciclovir is initiated to ablate transferred cells (see below). All patients receiving therapy on this study receive routine follow-up for the first year through contact with their COH HCT physician to identify any late complications due to infusion of gene-modified universal genetically modified UCBT. As non-malignant CD19⁺ B-cells may be subject to recognition by re-directed CTL, the persistence of the adoptively transferred CD19-specific CTL has the potential to cause B-cell immunodeficiency. Therefore, laboratory tests that reflect B-cell function are conducted by measuring serum immunoglobulin levels and determining the percentage of circulating B-cells until they normalize. If hypogammaglobulinemia becomes clinically significant, research participants are given intravenous immunoglobulin (IVIG) as replacement therapy until B-cell function returns. If by Day +100 Q-PCR detection of transferred T cells indicates continued persistence and patients require IVIG support, then ganciclovir ablation of T cells is instituted.

Timing/Criteria to Infuse T Cells

Research participants with an ANC >500 mm³, Karnofsky/Lansky ≧50, absence of infection, and who are not receiving ganciclovir, qualify for an infusion of the T cell product. T cells are infused immediately upon thawing a cryopreserved dose at the bedside. One of the most common infusion-related adverse events in adoptive immunotherapy trials is pulmonary toxicity (Table 2). Based on this experience, a T cell infusion is delivered in two parts. Initially, 10% of the T cell dose is infused and patients monitored. If there is no grade >2 (CTC vs. 3) adverse pulmonary toxicity attributed to the T cells over a 48-hour observation period, the remainder of the T cell product for the assigned dose-level is infused.

T Cell Dose Escalation De-Escalation Plan

Cohorts of three consecutively-enrolled subjects are assigned, in an ordered stratification to dose-level I (10⁸/m²), then dose-level II (10⁹/m²), followed by dose-level III (10 ¹⁰/m²). Dose escalation for a cohort may occur once 3 subjects have completed at least 28 days of post-infusion observation at a given dose-level. Dose escalation for a cohort is not permitted if within 28 days of a T cell infusion, two of the three research participants for a given Dose Level develops a new adverse event of grade ≧3 involving GVHD, cardiopulmonary, hepatic (excluding albumin), neurologic, or renal CTC vs. 3 parameters that is probably or definitely attributed to the infused T cell products. Should Adverse Events/toxicities be observed that result in cessation of treatment of patients at that dose-level/result in failure to met criteria for cohort dose escalation, three additional patients are treated at the prior Dose Level (FIG. 12 summarizes this plan).

Protocol Stopping Rules

The primary objective of the trial is to assess the feasibility of this treatment approach and acquisition of preliminary safety data. To help insure subject-safety, stopping rules are in place should excessive toxicity related to the T cell infusions be observed. The trial is halted if (i) more than 2 patients experience grade ≧3 GVHD within 100 days of a T cell infusion, (i) if the incidence of mortality is >30% at 100 days after enrolling 3 patients, or (iii) if the incidence of ineligibility to proceed with adoptive therapy reaches 75% after enrolling nine patients. The study is also halted if (i) a grade 5 adverse event probably or definitely attributed to the infuised T cells occurs in a research participant within 28 days of a T cell infusion, (ii) an incidence of grade 4 adverse event probably or definitely attributed to the infused T cells occurs in more than two research participants within 28 days of a T cell infuision, (iii) any patient receiving ganciclovir±systemic corticosteroids for ablation of T cells does not show an improvement to a toxicity grade of <3 within 14 days.

Ganciclovir Ablation to Resolve Toxicities Attributable to T Cell Infusion

The genetic modification of T cells to express the TK gene for the purposes of ganciclovir-induced in vivo ablation has been most extensively applied as a strategy to control the persistence of infused donor lymphocytes causing GVHD following allogeneic HCT (Bordignon et al., 1995; Cohen et al., 2000; Cohen et al., 2001; Litvinova et al., 2002; Verzeletti et al., 1998). This experience has demonstrated that expression of TK does not per se have a detrimental effect on T cell physiology while ganciclovir administration to patients experiencing GVHD following donor lymphocyte infusions of TK-expressing T cells is generally effective at ablating T cells in human hosts and aborting GVHD. The co-expression of HyTK in the CD19-specific CTL used in this Pilot/Phase I trial is justified based on the unknown incidence and severity of toxicities that the adoptive immunotherapy regimen may evoke for a particular research participant. Ablation of the infused T cells with ganciclovir will occur if: (1) subjects not taking rhIL-2 experience a grade 4 adverse event with an attribution to T cell therapy of >3 (likely or definitely), (2) within 36-hours of stopping rhIL-2 an adverse event does not improve to grade <3. Intravenous ganciclovir is used at 10 mg/kg/day divided between two doses with adjustments made for abnormal renal function. A 14-day course is prescribed, but this may be extended should symptomatic resolution is not achieved within this initial time interval. If symptoms do not respond to ganciclovir within 72 hours of initiating this therapy, or toxicities are severe, then additional immunosuppressive agents may be added at the discretion of the Principal Investigator. Furthermore, if toxicities are severe then additional immunosuppressive agents, such as corticosteroids, may be added earlier. If ablation is needed, then Q-PCR is used to assess the persistence of infused T cells from samples taken every two days from peripheral blood and weekly from bone marrow, until clearance of the genetically modified T cells is established.

Safety of Administering Low Dose rhIL-2 Following Infusion of Universal T Cells

In vivo persistence studies involving adoptively transferred ex vivo expanded gene-marked T cell lines specific for Epstein-Barr virus (EBV) have demonstrated the presence of cells in the circulation of transplant recipients in excess of 4 months and as long as 38 months following adoptive transfer (Heslop et al., 1996). Prior studies (Walter et al., 1995) analyzing the persistence of adoptively transferred CD8⁺ CMV-specific CTL clones in BMT recipients have demonstrated that cultured T cells can provide long term (>3 mo) immunity. The duration of persistence in these studies was found to correlate with the status of endogenous CMV-specific CD4⁺ T cell immunity; CD8⁺ clones did not persist long term in individuals without detectable CD4⁺ CMV-specific help. Infused CD19R⁺ CD8⁺ CTL are not likely to be supported by an endogenous CD19-specific CD4⁺ helper response and, in an analogous fashion to the CMV setting, these clones would not be expected to persist for prolonged periods of time following reinfusion. The administration of rhIL-2 to study subjects may support the in vivo expansion and persistence of infused genetically modified CD8⁺ UCBT clones as well as oligoclonal/polyclonal lines devoid or deficient in CD19R⁺ T_{HC}1 D4⁺ T cells. Following subcutaneous administration, rhL-2 exhibits a serum half-life of between 3-12 hours, sustained serum levels of 10-25 U/mL, and receptor saturating serum concentrations of 22 pM after an injection of 250,000 U/m².

Based on these considerations, patients are eligible, beginning 3 hours after the 9/10^{th} dosing of the T cell infusion for exogenous subcutaneous low-dose (5×10⁵ IU/m²/dose q 12-hrs) rhIL-2 given over 14 consecutive days, provided that subject there is no new Grade III or higher adverse event attributed to the infused T cells. The administration of rhIL-2 is stopped early if a new grade ≧3 adverse event is observed involving GVHD, cardiopulmonary, hepatic, neurological, or renal CTC vs. 3 parameters occurs with attribution to rhIL-2 of >3.

Statistical Considerations

The use of three patients per dose level is based on the utility of this design in numerous phase I trials of potentially toxic therapies at our institution and worldwide. The sample size is not based on formal statistical inferences, so there are no power calculations. The analysis plan includes calculation of summary descriptive statistics of patient characteristics, disease characteristics, observed toxicities, transplant engraftment parameters, clinical data sets such as absolute lymphocyte counts and survival time. Overall survival and progression-free survival is estimated using Kaplan-Meier curves. Exact binomial 95% confidence intervals for proportions surviving to fixed times are used as follow-up becomes complete. Repeated measures analysis of variance or univariate contrasts may be used to test for differences in selected clinical parameters over time. The primary objectives of these exploratory statistical analyses are to provide sufficient preliminary data necessary to properly design subsequent larger Phase VIII clinical trials. All statistical analyses are performed using JMP Version 5.1 and SAS Version 9.0 statistical software (SAS Institute Inc, Cary, N.C.).

### Example 15

### Analyses Conducted During the Trial

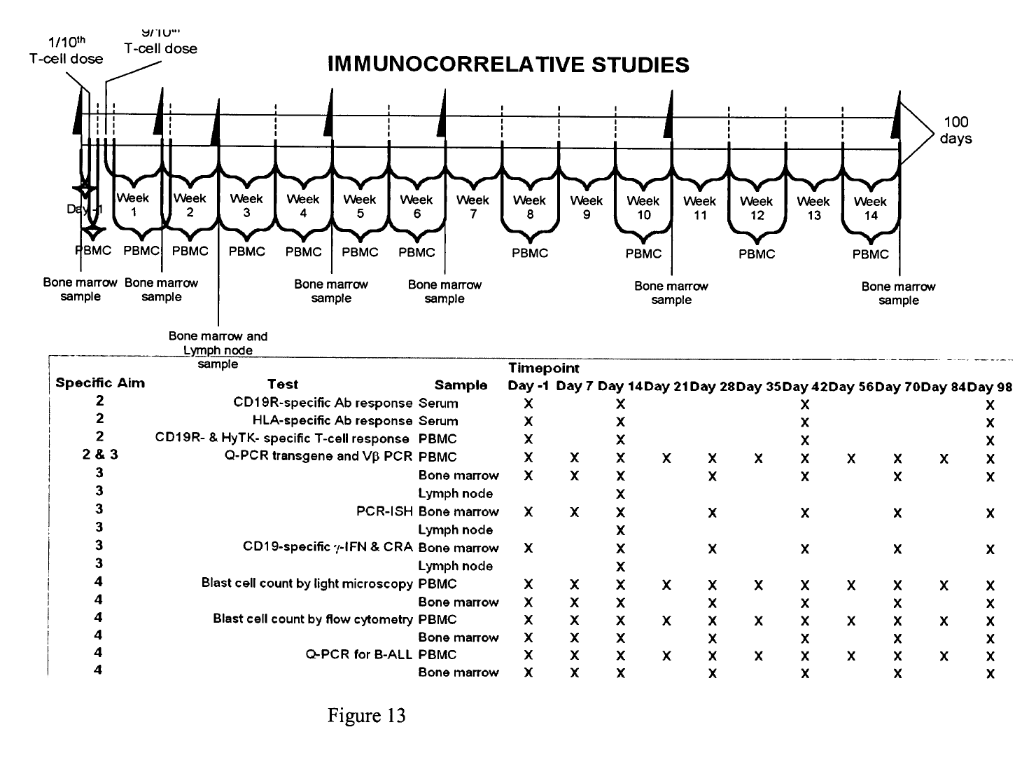

The protocol for the trial is designed to derive insights into several key issues pertaining to adoptive transfer of universal genetically modified CD19-specific UCBT in humans. The correlative studies described in this example seek to delineate the magnitude and duration of transferred T cell engraftment at low (10⁸/m²) versus high (10¹⁰/m²) cell doses. A secondary endpoint to be evaluated is whether an anti-transgene or allo-immune response occurs in these patients. FIG. 13 presents an overview of the timeline for repeated pre- and post-infusion patient-specific sample procurement. Analyses that involve quantifying the frequency of infuised T cells are evaluated using PCR- and flow-based approaches and peripheral blood collected from research participants at defined time points post infusion. Analyses evaluating antibody responses to the CD19R are evaluated by flow cytometry. Analyses involving detecting a cellular anti-transgene response are evaluated using in vitro T cell expansion and effector assays. Additional analyses depend on the presence of adequate numbers of infused cells in samples, and include the application of flow cytometry-based approaches to evaluate the phenotype and functional status of universal CD19-specific UCBT identified in specimens of peripheral blood and the evaluation of alternate mechanisms that may limit the persistence of T cells.

Analysis of Whether the Infusion of Low Versus High Doses Affect the Magnitude and Duration of Persistence of Transferred CD19-Specific Universal T Cells

The magnitude of expansion and duration of persistence for infused universal genetically modified UCBT in serially acquired peripheral blood samples is examined by Q-PCR using a primer pair that specifically amplifies the unique CD19R chimeric transgene (as described above). Using this methodology, CD19R⁺ T cells spiked into peripheral blood that comprise as few as 1/50,000 of PBMC can be routinely detected. The specificity of this assay is 100% and no false positives have been identified to date in control samples. This assay provides persistence data in the absence of in vivo-expansion, such as might arise following the low-dose infusions. The Vβ TCR used by the universal UCBT clone is identified and the percentage and pattern of Vβ TCR-usage is followed before and after adoptive transfer.

The persistence of modified T cells in peripheral blood is determined by quantitative PCR (Q-PCR) using the TaqMan fluorogenic 5′ nuclease reaction (Fabb et al., 1997; Gal et al., 2004; Heim et al., 2003) with genomic DNA isolated from patient peripheral blood samples at each time point (FIG. 13). Q-PCR has been validated as an extremely sensitive and accurate approach to quantitate DNA in blood samples (Gal et al., 2004; Heim et al., 2003; Sanchez and Storch, 2002; Stirewelt et al., 2001). These analyses quantify the presence of the transgene DNA sequence integrated in the genome of modified T cells. Using the primer sets described below, the Q-PCR assay has been developed and implemented to detect and quantify T cells that contain plasmid vectors, and are sensitive enough to reliably detect at least one vector-containing cell in 50,000 PBMC. This assay is currently being used to track the persistence of gene-modified clones in the circulation of research participants in an adoptive therapy trial for leukemia. The primer pair used to detect the integrated transgene is 5′ HcFc (5′ TCTTCCTCTA CACAGCAAGCTCACCGTGG 3′ (SEQ ID NO:11)) and 3′Huζ (5′ GAGGGTTCTTCCTTCT CGGCTTTC 3′ (SEQ ID NO:12)). These primers amplify a 360 basepair fragment spanning the Fc-CD4-TM-ζ sequence fusion site that is present in the chimeric constructs that is detected with the TaqMan hybridization probe FAM—5′-TTCACTCTGAAGAAGATGCCTAGCC-3′—TAMRA (FAM—SEQ ID NO:13—TAMRA). The primer pair used to detect the human β-globin gene is Pco3 (5′ ACACAACTGTGTTCACTAGC 3′ (SEQ ID NO:14)) and GII (5′ GTCTCCTTAAACCTGTCTTG 3′ (SEQ ID NO:15)) that is detected with the Taqman hybridization probe for β-globin is HEX—5′-ACCTGACTCCTGAGGAGAAGTCT-3′—TAMRA (HEX—SEQ ID NO:16—TAMRA). Each PCR amplification reaction is performed in triplicate. The average threshold value from the CD19R amplification is used to determine the ratio of CD19R⁺ transgene cells/total cells, and the average threshold value from the β-globin amplification is used to normalize for template amplification inconsistencies. The absolute number of CD19R⁺ cells at each time point is then calculated based on the total number of cells/sample. These analyses allow the determination of both the persistence of genetically modified T cells in PBMC in the recipient.

Transferred Universal CD19-Specific UCBT Persistence is Assessed by TCR V/β Spectratyping

TCR Vβ PCR spectratyping is used to evaluate the survival of infused T cells with serially acquired peripheral blood specimens of patients receiving the universal T cell product. TCR Vβ spectratyping quantifies the T cell complexity of a cell population by measuring the complementary determining region 3 (CDR3) length complexity in mRNA samples derived from cells to be evaluated (Sprent and Surh, 2003; Sloand et al., 2002). Because each Vβ family has a unique CDR3 length, spectratyping allows a determination of the frequency for individual Vβ chains in a T cell population. TCR Vβ spectratyping is used to evaluate the T cell complexity in samples obtained from the research participant at each of the peripheral blood sampling time points indicated and this is compared with the Vβ used by the infused T cell clone, as measured in Test Panel C (Table 1, FIG. 2). This RT-PCR method amplifies CDR3 regions from 46 known functional TCR Vβ subfamilies. Each cDNA is evaluated in a series of 5 PCR reactions, with each reaction containing a single TCR Vβ constant region primer tagged with the fluorescent FAM (6-carboxyfluorescein) dye, and a mixture of 4 to 7 specific primers specific for individual Vβ chains. The TCR Vβ expression results are reported as percentage of CD8+and/or CD3⁺ T cells in the sample.

Determination of Any Potential Attenuated T Cell Persistence

An immune response directed against the infused universal CD19-specific T cells may be due to T cell and/or antibody recognition of (i) foreign HLA antigens, due to incomplete suppression of classical HLA class I/II gene expression and/or (ii) immunogenic transgenes. Therefore, all infused research participants are studied for evidence of priming of anti-CD19R, anti-HyTK, and allo-immune CTL-responses and anti-HLA class I/II and anti-CD19R antibody-responses using approximately 10 mL of recipient PBMC and 5 mL recipient serum obtained pre-infusion and on days ⁺14, ⁺42, and ⁺98 after T cell infusion (FIG. 13). To detect T cell allo-immune responses (directed against disparate HLA molecules remaining on universal T cells), recipient-specific responder T cells derived from PBMC are incubated with ratios of irradiated universal CD19-specific stimulator T cells in a mixed lymphocyte response (MLR). T cell responses against the infused universal T cells are detected by specific uptake of ³H-thymidine.

To detect CTL anti-transgene responses, the cryopreserved universal CD19-specific T cell product and PBMC isolated from recipient peripheral blood after the T cell infusion are repetitively stimulated at 7-day intervals in separate cultures using irradiated autologous (patient-specific) T cells, transfected with CD19R and HyTK genes and which act as antigen presenting cells. After 3 stimulation cycles, T cells are screened by CRA against ⁵¹Cr-labeled patient-derived LCL, CD19R⁺ LCL, HyTK⁺ LCL, and CD19R+HyTK⁺ LCL. These targets differentiate the specificity of the recipient's immune response between reactivity against HyTK and the chimeric immunoreceptor. As a positive control for immunocompetency, EBV-specific immune responses are evaluated in PBMC obtained from recipient using autlogous LCLs.

To study whether an antibody response develops against the cell-surface bound chimeric immunoreceptor, serum is drawn before and after T cell infusion (FIG. 13). This serum is tested for binding against a genetically modified Jurkat cell-clone expressing the CD19R gene. Background binding is defined by incubating the serum in parallel with unmodified Jurkat cells. Patient-derived antibody is detected by flow cytometry using FITC-conjugated mouse mAb specific for human Fab (which has been demonstrated not to cross-react with CD19R). Whether there are recipient antibody responses against HLA molecules remaining on the infused universal T cells is also evaluated. This assay is a core component of HLA-typing and is operational at COH. In brief, serum from the recipient at defined timepoints (FIG. 13) is evaluated for binding to the universal T cell clone. Surface bound antibody is detected by flow cytometry using fluorescent anti-human Ig.

Statistical Considerations

Statistical analyses is carried out on these correlative data sets. The analysis of the pilot study is descriptive in nature, but more formal analyses are planned when the sample is ultimately expanded in a Phase II trial. The primary endpoint is based on persistence (defined as # gene-modified T cells/50,000 peripheral blood PBMCs) of adoptively transferred genetically-modified T cell lines and clones in the circulation, as assessed by CD19R/HyTK-pMG specific Q-PCR analysis of serially acquired peripheral blood. Repeated-measure linear models are applied to estimate the frequency of transferred T cell counts over time among the sequential peripheral blood samples. Adjustments are made for additional explanatory variables in our model, including the dose of infused cells, infusion of a defined T cell clone versus infusion of oligoclonal/polyclonal T cell line containing both CD8⁺ and CD4⁺ T cells, and administration of exogenous rhIL-2. Logarithms of the count data, or work with a log-link in the context of a generalized linear model, are taken to better fit with working assumptions of additive effects and Gaussian errors. Our summary comparisons include the Kruskal-Wallis test to compare experimental PCR and flow cytometry results to baseline and controls.

To answer the question whether there a selective survival advantage for a sub-population of T cells within an infused T cell line, expression patterns are compared between the TCR Vβ usage from in vivo-sampling to the expression patterns of TCR Vβ usage on archived T cell populations used for infusion. Comparisons involve graphical display of Vβ usage profiles, Hotelling's T² test and related multivariate methods, as well as univariate analysis of Vβ diversity. To test for differences in the development of immune responses to transgenes expressed by infused cells, the mean differentiation of the specificity of the recipient's immune response between reactivity against hygromycin, TK and the chimeric immunoreceptor using Wilcoxon rank-sum tests are compared. 95% confidence intervals are included when appropriate.

### Example 16

### Evaluation of the Accumulation and Functional Status of Adoptively Transferred Universal CD19-Specific Genetically Modified T Cells Within a Tumor Microenvironment

Since the most common site of leukemic relapse for ALL is in the marrow and lymph node, it is likely that minimal residual leukemic disease also resides in these microenvironments. The clinical efficacy of targeting relapsed disease with adoptively transferred universal CD19-specific UCBT is therefore dependent, in part, on the efficiency of these cells to traffic to, and accumulate in, the marrow compartment and secondary lymphoid structures microenvironment. Moreover, once localized to these microenvironments these T cells need to be functionally intact for recognition and lysis of CD19⁺ targets. The number, composition, homing and functional status of marrow- and lymph node-residing universal CD19-specific T cells are evaluated in recipients using Q-PCR as well as flow cytometry-based methodologies. These studies are carried out using bone marrow aspirate specimens collected from research participants at defined time points post infusion (FIG. 13).

The numbers of transferred T cells that migrate to bone marrow and lymph node are quatified using plasmid vector-specific Q-PCR and TCR Vβ methodologies as described above. These data sets are matched to data sets derived from the same analyses on peripheral blood.

If the transferred T cells are able to migrate to lymph nodes, the anti-leukemia activity might be limited if the malignant cells are replicating within the bone marrow and/or lymph node architecture that is inaccessible to the genetically modified T cells. Therefore, the distributions of both the infused universal T cells and the ALL cells within the marrow and secondary lymphoid tissue are determined by PCR-ISH. Samples are obtained accordingly (FIG. 13). The paraffin-fixed tissues are processed as described above. Bone marrow specimens before adoptive immunotherapy and bone marrow and lymph node samples from untreated patients serve as negative controls. Digital microscopy and quantitative image analysis are used to describe the relationship between the genetically modified T cells and the tumor cells.

The ability of universal CD19-specific UCBT recovered from marrow aspirates and lymph node biopsy to be triggered through the anti-CD19 CAR is assessed by CD19-specific induction of γ-IFN gene expression by intracellular cytokine staining (ICS). As discussed above, chimeric CD19R⁺ UCBT express robust γ-IFN and cytolysis effector functions upon activation by CD19⁺ targets, and this is a release criteria for the subject-specific T cell product (Test Panel C, Table 1, FIG. 2). ICS has been shown to be a sensitive and quantitative assay to measure antigen-specific T cell effector functions, and, optimized protocols are employed to specifically measure γ-IFN content in the cytoplasm of T cells (Ghanekar and Maecker, 2003; Letsch and Scheibenbogen, 2003; Sloand et al., 2002). These ICS analyses are performed using aliquots of bone marrow samples and lymph node biopsy specimens obtained from subjects at the time points indicated (FIG. 13). These studies are possible in the absence of in vivo T cell expansion if ≧10⁴ infused T cells are present in sample to be analyzed, which would be achieved if ≧10% of the infused T cells localize to secondary lymphoid tissue and/or marrow at dose level I or ≧1% persist at dose level II or III.

Following depletion of red blood cells, samples are incubated for 5 hours with CD19⁺ stimulator cells in the presence of brefeldin A, and production of γ-IFN by universal CD19-specific UCBT are evaluated using ICS as per standard protocols using a PE-Cy7-conjugated anti-human γ-IFN antibody (BD Biosciences) (Kuzushima et al., 1999). In parallel, γ-IFN expression from unstimulated PBMCs isolated from patients, non-infused cryopreserved genetically modified universal UCBT product, and in vitro tumor-stimulated non-infused genetically modified T cell product is evaluated. To identify the specific effector cells producing γ-IFN samples are co-labeled with FITC-labeled anti-Fc (to identify genetically modified T cells), APC-Cy7-conjugated anti CD8 (to identify T cells), and if available, PE-conjugated Vβ-specific mAb, to identify the infused universal CD19-specific UCBT clone, and evaluated on a 6-color capable cytometer (BD FACS-Canto). These analyses provide important insights on the functional status of transferred universal CD19-specific UCBT localized to the two most common sites of MRD.

Transgene-specific Q-PCR frequencies in marrow and blood are compared by graphical methods and by multivariate test statistics. The percentage and median fluorescent intensity (MFI) of the bound fluorochromes by defined populations of “gated” T cells are determined using FCS Express flow cytometer analysis software. The coefficient of variation around the median is used to generate 95% confidence intervals. The percentage expression and MFI are compared between gated T cell subpopulations (clones, lines, archived specimens, samples recovered from peripheral blood and bone marrow). Differences between these T cell populations are presented in histogram format for defined T cell gated populations and the Kruskal-Wallis test determines if there is statistical difference between percentage expression and MFI at the 90%, 95% and 99% confidence level.

### Example 17

### Evaluation of the Anti-Tumor Activity of Adoptively Transferred Universal CD19-Specific Genetically Modified T Cells

The development of approaches for detecting the capacity of transferred cells to cytoreduce/eradicate tumor cells of ALL in vivo is a high priority. Since the recipients in this trial are in relapse at the time of adoptive immunotherapy, the disease burden before and after T cell infusion is quantified (Pui et al., 2004). Three approaches are used: (i) morphologic inspection and quantification of blast burden in peripheral blood and bone marrow, (ii) Q-PCR using ALL-specific PCR amplimers to quantify levels of marrow and peripheral blood MRD, relative to marrow numbers of universal CD19-specific UCBT, and (iii) multiparameter flow cytometry to detect aberrant antigen expression. These studies provide a dynamic view of the kinetics of blast eradication, relative to the numbers of infused anti-tumor effectors. Repeated sampling from peripheral blood and bone marrow (FIG. 13) help to minimize sampling errors.

Measurement of blast percentage are performed by expert morphologists who are part of the pathology department at COH. However, this conventional technique cannot detect B-ALL when there are fewer than approximately 1010 total cells. If the original B-ALL cell carries a molecular or antigenic marker that distinguishes it from non-leukemic cells, then all cells of the leukemic clone exhibit the same marker. This property allows the application of sensitive new techniques that use either PCR or antibody to detect or quantify leukemic cells. Competitive PCR-based methods can detect and quantify the number of cells with clonal rearrangements (with a limit of detection of 10⁻⁴ to 10⁻⁵). Therefore, to quantify ALL blast burden, studies are conducted to develop and apply the Q-PCR assays. In some cases a leukemia-specific mutation is not evident. Therefore, multiparameter flow cytometry is used to detect combinations of surface antigens that are semispecific for the B-ALL clone and thereby quantify residual disease (to a level of approximately 10⁻⁴).

Pre-treatment and post-treatment assays are summarized and standard non-parametric methods are used to test comparisons. Linear models are used to relate changes in disease burden to assays of the abundance and localization of universal CD19-specific UCBT. A simple Bonferroni correction is used to adjust p-values for the simultaneous testing of multiple outcomes based on morphology, QT-PCR and flow cytometry.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive. It will also be appreciated that in this specification and the appended claims, the singular forms of “a,” “an” and “the” include plural reference unless the context clearly dictates otherwise. It will further be appreciated that in this specification and the appended claims, The term “comprising” or “comprises” is intended to be open-ended, including not only the cited elements or steps, but further encompassing any additional elements or steps.

### BIBLIOGRAPHY

Adams, E. J. and P. Parham (2001). Genomic analysis of common chimpanzee major histocompatibility complex class I genes. Immunogenetics 53(3):200-208.

Altenschmidt, U. et al. (1997). Adoptive transfer of in vitro-targeted, activated T lymphocytes results in total tumor regression. J Immunol 159:5509-5515.

Bahramian, M. B. and H. Zarbul (1999). Transcriptional and Posttranscriptional Silencing of Rodent alpha1(I) Collagen by a Homologous Transcriptionally Self-Silenced Transgene. Mol and Cell Biol 19(1):274-283.

Berenson, J. R. et al. (1975). Syngeneic adoptive immunotherapy and chemoimmunotherapy of a Friend leukemia: requirement for T cells. J Immunol 115(1):234-238.

Berger, C. et al. (2000). Expression of herpes simplex virus ICP47 and human cytomegalovirus US 11 prevents recognition of transgene products by CD8(+) cytotoxic T lymphocytes. J Virol 74(10):4465-4473.

Bird, R. E. et al. (1988). Single-chain antigen-binding proteins. Science 242:423-426.

Bordignon, C. et al. (1995). Transfer of the HSV-tk gene into donor peripheral blood lymphocytes for in vivo modulation of donor anti-tumor immunity after allogeneic bone marrow transplantation. Hum Gene Ther 6:813-819.

Bosher, J.M. et al. (1999). RNA Interference Can Target Pre-mRNA: consequences for Gene Expression in a Caenorhabditis elegans Operon. Genetics 153:1245-1256.

Brocker, T. and K. Kaijalainen (1998). Adoptive tumor immunity mediated by lymphocytes bearing modified antigen-specific receptors. Adv Immunol 68:257-269.

Brodie, S. J. et al. (1998). The effects of pharmacological and lentivirus-induced immune suppression on orbivirus pathogenesis: assessment of virus burden in blood monocytes and tissues by reverse transcription-in situ PCR. J Virol 72(7):5599-5609.

Brummelkamp, T. R. et al. (2002). A system for stable expression of short interfering RNAs in mammalian cells. Science 296:550-553.

Butler, S. L. et al. (2001). A quantitative assay for HIV DNA integration in vivo. Nat Medicine 7(5):631-634.

Castanotto, D. et al. (2002). Functional siRNA expression from transfected PCR products. Rna 8(11):1454-1460.

Chan, A. et al. (1991). Irving BA, Fraser JD, Weiss A:, The zeta chain is associated with a tyrosine kinase and upon T-cell antigen receptor stimulation associates with ZAP-70, a 70-kDa tyrosine phosphoprotein. Proc Natl Acad Sci USA 88:9166-9170.

Cohen, J. et al. (2000). Preservation of graft-versus-infection effects after suicide gene therapy for prevention of graft-versus-host disease. Hum Gene Ther 11:2473-2481.

Cohen, J. et al., (2001). Suicide gene therapy of graft-versus-host disease: immune reconstitution with transplanted mature T cells. Blood 98:2071-2076.

Cooper, L. J. et al. (2003). T-cell clones can be rendered specific for CD19: toward the selective augmentation of the graft-versus-B-lineage leukemia effect. Blood 101(4):161637-44.

Cooper, L. J. et al. (2004). Enhanced transgene expression in quiescent and activated human CD8⁺ T cells. Hum Gene Ther 15(7):648-658.

Correia-Neves, M. et al. (2001). The shaping of the T cell repertoire. Immunity 14(l):21-32.

Dinndorf, P. et al. (2001). Phase I trial of anti-B4-blocked ricin in pediatric patients with leukemia and lymphoma. J Immunother 24:511-516.

Eberlein, T. J. et al. (1982). Regression of a disseminated syngeneic solid tumor by systemic transfer of lymphoid cells expanded in interleukin 2. J Exp Med 156(2):385-397.

Echeverri, C. et al. (2002). Immunophenotypic variability of B-cell non-Hodgkin lymphoma: a retrospective study of cases analyzed by flow cytometry. Am J Clin Pathol 117(4):615-620.

Eshhar, Z. (1997). Tumor-specific T-bodies: towards clinical application. Cancer Immunol Immunother 45:131-136.

Eshhar, Z. et al. (1993). Specific activation and targeting of cytotoxic lymphocytes through chimeric single chains consisting of antibody-binding domains and the gamma or zeta subunits of the immunoglobulin and T-cell receptors. Proc Natl Acad Sci USA 90:720-724.

Fabb, S. A., et al. (1997) Generation of novel human MHC class II mutant B-cell lines by integrating YAC DNA into a cell line homozygously deleted for the MHC class II region. Hum Mol Genet 6(8):1295-304.

Fire, A. et al. (1998). Potent and specific genetic interference by double stranded RNA in Caenorhabditis elegans. Nature 391:806-811.

Foon, K. et al. (1986). Recent advances in the immunologic classification of leukemia. Semin Hematol 23:257-283.

Freitas, A. A. et al. (1996). Cellular competition modulates survival and selection of CD8+ T cells. Eur J Immunol 26(11):2640-2649.

Gal, S. et al. (2004). Quantitation of circulating DNA in the serum of breast cancer patients by real-time PCR. Br J Cancer 90:1211-1215.

Goldrath, A. W. and M. J. Bevan (1999). Selecting and maintaining a diverse T-cell repertoire. Nature 402(6759):255-262.

Geurts, A. M. et al. (2003). Gene transfer into genomes of human cells by the sleeping beauty transposon system. Mol Ther 8(1):108-117.

Ghanekar, S. and H. T. Maecker (2003). Cytokine flow cytometry: multiparametric approach to immune function analysis. Cytotherapy 5:1-6.

Gonzalez, S. et al. (2005). Amplification of RNAi-Targeting HLA mRNAs. Mol Ther 11(5):811-818.

Goulden, N. et al. (2003). Minimal residual disease prior to stem cell transplant for childhood acute lymphoblastic leukaemia. Br J Haematol 122:24-29.

Greenberg, P.D. (1986). Therapy of murine leukemia with cyclophosphamide and immune Lyt-2+cells: cytolytic T cells can mediate eradication of disseminated leukemia. J Immunol 136:1917-1922.

Hekele, A. et al. (1996). Growth retardation of tumors by adoptive transfer of cytotoxic T lymphocytes reprogrammed by CD44v6-specific scFv:zeta-chimera. Int J Cancer 68:232-238.

Heim, A. et al. (2003). Rapid and quantitative detection of human adenovirus DNA by real-time PCR. J Med Virol 70:228-239.

Heslop, H. E. and C. M. Rooney (1997). Adoptive cellular immunotherapy for EBV lymphoproliferative disease. Immunol Rev 157:217-222.

Heslop, H. et al. (1996). Long-term restoration of immunity against Epstein-Barr virus infection by adoptive transfer of gene-modified virus-specific T lymphocytes. Nat Medicine 2:551-555.

Hu, H. M. et al. (2002). Development of antitumor immune responses in reconstituted lymphopenic hosts. Cancer Res 62(14):3914-3919.

Hulkkonen, J. et al. (2002). Surface antigen expression in chronic lymphocytic leukemia: clustering analysis, interrelationships and effects of chromosomal abnormalities. Leukemia 16(2):178-185.

Irving, B. and A. Weiss (1991). The cytoplasmic domain of the T cell receptor zeta chain is sufficient to couple to receptor-associated signal transduction pathways. Cell 64:891-901.

Izsvak, Z. et al. (2000). Sleeping Beauty, a wide host-range transposon vector for genetic transformation in vertebrates. J Mol Biol 302(1):93-102.

Jensen, M. et al. (1998). CD20 is a molecular target for scFvFc:zeta receptor redirected T cells: implications for cellular immunotherapy of CD20+malignancy. Biol Blood Marrow Transplant 4:75-83.

Kawasaki, H. and K. Taira (2004). Induction of DNA methylation and gene silencing by short interfering RNAs in human cells. Nature 431:211-217.

Kim, D. W. et al. (1990). Use of the human elongation factor 1 alpha promoter as a versatile and efficient expression system. Gene 91:217-223.

Kuzushima, K. et al. (1999). Rapid determination of Epstein-Barr virus-specific CD8(+) T-cell frequencies by flow cytometry. Blood 94:3094-3100.

Lanier, L. L. (1998). NK cell receptors. Annu Rev Immunol 16:359-393.

Lawson, T. et al. (2001). Influenza A antigen exposure selects dominant Vbetal7+TCR inhuman CD8⁺ cytotoxic T cell responses. Int Immunol 13:1373-1381.

LeBien, T. W. (2000). Fates of human B-cell precursors. Blood 96(1):9-23.

Lee, N. et al. (1998). HLA-E surface expression depends on binding of TAP-dependent peptides derived from certain HLA class I signal sequences. J Immunol 160(10):4951-4960.

Lee, N. S. et al. (2002). Expression of small interfering RNAs targeted against HIV-1 rev transcripts in human cells. Nat Biotech 20:500-505.

Letsch, A. and C. Scheibenbogen (2003). Quantification and characterization of specific T-cells by antigen-specific cytokine production using ELISPOT assay or intracellular cytokine staining. Methods 31:143-149.

Li, C. R. et al. (1994). Recovery of HLA-restricted cytomegalovirus (CMV)-specific T-cell responses after allogeneic bone marrow transplant: correlation with CMV disease and effect of ganciclovir prophylaxis. Blood 83(7):1971-1979.

Litvinova, E. et al. (2002). Graft-versus-leukemia effect after suicide-gene-mediated control of graft-versus-host disease. Blood 100: 2020-2025.

Liu, G. et al. (2004). Excision of Sleeping Beauty transposons: parameters and applications to gene therapy. J Gene Med 6(5):574-583.

Ljunggren, H. G. and K. Karre (1990). In search of the ‘missing self’: MHC molecules and NK cell recognition. Immunol Today 11(7):237-244.

Longo, D. L. et al. (2000). Combination chemotherapy followed by an immunotoxin (anti-B4-blocked ricin) in patients with indolent lymphoma: results of a phase II study. Cancer J6:146-150.

Lopes de Menezes, D. E. et al. (2000). Selective targeting of immunoliposomal doxorubicin against human multiple myeloma in vitro and ex vivo. Biochim Biophys Acta 1466:205-220.

Lupton, S. D. et al. (1991). Dominant positive and negative selection using a hygromycin phosphotransferase-thymidine kinase fusion gene. Mol Cell Biol 11(6):3374-3378.

Ma, D. et al. (2002). Radioimmunotherapy for model B cell malignancies using 90Y-labeled anti-CD19 and anti-CD20 monoclonal antibodies. Leukemia 16:60-66.

Mackall, C. L. et al. (1996). Thymic-independent T cell regeneration occurs via antigen-driven expansion of peripheral T cells resulting in a repertoire that is limited in diversity and prone to skewing. J Immunol 156(12):4609-4616.

Mackall, C. L. et al. (1997). Restoration of T-cell homeostasis after T-cell depletion. Semin Imiunol 9(6):339-346.

Maine, G. N. and J. J. Mule (2002). Making room for T cells. J Clin Invest 110(2):157-159.

Marrack, P., et al. (2000). Homeostasis of alpha beta TCR⁺ T cells. Nat Immunol 1(2):107-111.

Marsh, S. G. et al. (2002). Nomenclature for factors of the HLA system, 2002. Tissue Antigens 60(5):407-464.

McGuinness, R. et al. (1999). Anti-tumor activity of human T cells expressing the CC49-zeta chimeric immune receptor. Hum Gene Ther 10:165-173.

Mitchell, P. et al. (2003). Targeting primary human Ph(+) B-cell precursor leukemia-engrafted SCID mice using radiolabeled anti-CD19 monoclonal antibodies. J Nucl Med 44:1105-1112.

Montgomery, M. K. et al. (1998). RNA as a target of double-stranded RNA-mediated genetic interference in Caenorhabditis elegans. Proc Natl Acad Sci USA 95:15502-15507.

Moss, P. et al. (1991). Extensive conservation of alpha and beta chains of the human T-cell antigen receptor recognizing HLA-A2 and influenza A matrix peptide. Proc Natl Acad Sci USA 88:8987-8990.

Morris, K. V. et al. (2004). Small Interfering RNA-Induced Transcriptional Gene Silencing in Human Cells. Science 305:1289-1292.

Nishikura, K. (2000). A short primer on RNAi: RNA-directed RNA polymerase acts as a key catalyst. Cell 107:415-418.

Ngo, H. et al. (1998). Double-stranded RNA induces mRNA degradation in Trypanosoma brucei. Proc Natl Acad Sci USA 95:14687-14692.

Ohlfest, J. R. et al. (2004). Integration and long-term expression in xenografted human glioblastoma cells using a plasmid-based transposon system. Mol Ther 10(2):260-8.

Pal-Bhadra, M. et al. (2002). RNAi related mechanisms affect both transcriptional and posttranscriptional transgene silencing in drosophila. Mol Cell 9:315-327.

Philip, T. and P. Biron (1991). Role of high-dose chemotherapy and autologous bone marrow transplantation in the treatment of lymphoma. Eur J Cancer 27:320-322.

Plasterk, R. H. et al (1999). Resident aliens: the Tc1/mariner superfamily of transposable elements. Trends Genet 15(8):326-332.

Pui, C. (1995). Childhood leukemias. N Engl J Med 332:1618-1630.

Pui, C. et al. (2004). Acute lymphoblastic leukemia. N Engl J Med 350:1535-1548.

Ramaswamy, G. and F.J. Slack (2002). siRNA. A guide for RNA silencing. Chem Biol 9(10):1053-1055.

Riddell, S. R. and P. D. Greenberg (1990). The use of anti-CD3 and anti-CD28 monoclonal antibodies to clone and expand human antigen-specific T cells. J Immunol Methods 128(2):189-201.

Roy, D. C. et al. (1995). Elimination of B-lineage leukemia and lymphoma cells from bone marrow grafts using anti-B4-blocked-ricin immunotoxin. J Clin Immunol 15:51-57.

Sanchez, J. and G. A. Storch (2002). Multiplex, quantitative, real-time PCR assay for cytomegalovirus and human DNA. J Clin Microbiol 40:2381-2386.

Sapra, P. et al. (2004). Improved therapeutic responses in a xenograft model of human B lymphoma (Namalwa) for liposomal vincristine versus liposomal doxorubicin targeted via anti-CD19 IgG2a or Fab′ fragments. Clin Cancer Res 10:1100-1111.

Sharp, P. A. (2001). RNA interference. Genes and Development 15:485-490.

Shen, X. and R. Konig (2001). Post-thymic selection of peripheral CD4+T-lymphocytes on class II major histocompatibility antigen-bearing cells. Cell Mol Biol (Noisy-le-grand) 47(1):87-96.

Sijen, T. et al. (2001). Transcriptional and posttranscriptional gene silencing are mechansitically related. Curr Biol 11:436-440.

Sloand, E. et al. (2002). Intracellular interferon-gamma in circulating and marrow T cells detected by flow cytometry and the response to immunosuppressive therapy in patients with aplastic anemia. Blood 100:1185-1191.

Sprent, J. and C. D. Surh (2003). Cytokines and T cell homeostasis. Immunol Lett 85(2):145-149.

Stamenkovic, I. and B. Seed (1988). CD19 the earliest differentiation antigen of the B cell lineage, bears three extracellular immunoglobulin-like domains and an Epstein-Barr virus-related cytoplasmic tail. J Exp Med 168:1205-1210.

Stirewalt, D. et al. (2001). Quantitative, real-time polymerase chain reactions for FLT3 internal tandem duplications are highly sensitive and specific. Leuk Res 25:1085-1088.

Szatrowski, T. et al. (2003). Lineage specific treatment of adult patients with acute lymphoblastic leukemia in first remission with anti-B4-blocked ricin or high-dose cytarabine: Cancer and Leukemia Group B Study 9311. Cancer 97:1471-1480.

Tanchot, C. et al. (1997). Lymphocyte homeostasis. Semin Immunol 9(6):331-337.

Tsimberidou, A. et al. (2003). Anti-B4 blocked ricin post chemotherapy in patients with chronic lymphocytic leukemia—long-term follow-up of a monoclonal antibody-based approach to residual disease. Leuk Lymphoma 44:1719-1725.

Verzeletti, S. et al. (1998). Herpes simplex virus thymidine kinase gene transfer for controlled graft-versus-host disease and graft-versus-leukemia: clinical follow-up and improved new vectors. Hum Gene Ther 9:2243-2251.

Volpe, T. A. et al. (2002). Regulation of Heterchromatic Silencing and Histone H3 Lysine-9 Methylation by RNAi. Science 297:1833-1837.

Walter, E. A. et al. (1995). Reconstitution of cellular immunity against cytomegalovirus in recipients of allogeneic bone marrow by transfer of T-cell clones from the donor. N Engl J Med 333(16):1038-1044.

Wassenegger, M. (2000). RNA-directed DNA methylation. Plant Mol Biol 43:203-220.

Wassenegger, M. et al. (1994). RNA-directed de novo methylation of genomic sequences in plants. Cell 76:567-576.

Yant, S. R. et al. (2004). Mutational Analysis of the N-Terminal DNA-Binding Domain of Sleeping Beauty Transposase: Critical Residues for DNA Binding and Hyperactivity in Mammalian Cells. Mol Cell Biol 24(20):. 9239-9247.

York, I.A. et al. (1994). A cytosolic herpes simplex virus protein inhibits antigen presentation to CD8⁺ T lymphocytes. Cell 77(4):525-535.

Zeng, Y. and B. R. Cullen (2002). RNA interference in human cells is restricted to the cytoplasm. RNA 8(7):855-860.

What is claimed is:

1. A genetically engineered T cell comprising stably incorporated in its genome a nucleic acid encoding a chimeric antigen receptor (CAR), one or more nucleic acids each encoding an RNAi molecule corresponding to a gene encoding an HLA class I gene and one or more nucleic acids each encoding an RNAi molecule corresponding to a gene encoding an HLA class II gene.

2. The genetically engineered T cell of claim 1 which further comprises a nucleic acid encoding a non-classical HLA gene stably incorporated in its genome.

3. The genetically engineered T cell of claim 2, wherein the non-classical HLA gene is an HLA E gene.

4. The genetically engineered T cell of claim 1 which further comprises a nucleic acid encoding a selection-suicide protein stably incorporated in its genome.

5. The genetically engineered T cell of claim 2 which further comprises a nucleic acid encoding a selection-suicide protein stably incorporated in its genome.

6. The genetically engineered T cell of claim 3 which further comprises a nucleic acid encoding a selection-suicide protein stably incorporated in its genome.

7. The genetically engineered T cell of claim 1, wherein the CAR is CD19R.

8. The genetically engineered T cell of claim 1, wherein the RNAi molecules corresponding to a gene encoding an HLA class I gene are an shRNA molecule and an siRNA molecule and wherein the RNAi molecules corresponding to a gene encoding an HLA class II gene are an shRNA molecule and an siRNA molecule.

9. The genetically engineered T cell of claim 7, wherein the RNAi molecules corresponding to a gene encoding an HLA class I gene are an shRNA molecule and an siRNA molecule and wherein the RNAi molecules corresponding to a gene encoding an HLA class II gene are an shRNA molecule and an siRNA molecule.

10. A process for making a genetically engineered T cell comprising:
(a) introducing a nucleic acid encoding a chimeric antigen receptor (CAR) into a T cell;
(b) introducing one or more nucleic acids each encoding an RNAi molecule corresponding to a gene encoding an HLA class I gene; and
(c) introducing one or more nucleic acids each encoding an RNAi molecule corresponding to a gene encoding an HLA class II gene.

11. The process of claim 10 which further comprises introducing a nucleic acid encoding a non-classical HLA gene.

12. The process of claim 11, wherein the non-classical HLA gene is an HLA E gene.

13. The process of claim 10 which further comprises introducing a nucleic acid encoding a selection-suicide protein.

14. The process of claim 11 which further comprises introducing a nucleic acid encoding a selection-suicide protein.

15. The process of claim 12 which further comprises introducing a nucleic acid encoding a selection-suicide protein.

16. The process of claim 10, wherein the CAR is CD19R.

17. The process of claim 10, wherein the RNAi molecules corresponding to a gene encoding an HLA class I gene are an shRNA molecule and an siRNA molecule and wherein the RNAi molecules corresponding to a gene encoding an HLA class II gene are an shRNA molecule and an siRNA molecule.

18. The process of claim 16, wherein the RNAi molecules corresponding to a gene encoding an HLA class I gene are an shRNA molecule and an siRNA molecule and wherein the RNAi molecules corresponding to a gene encoding an HLA class II gene are an shRNA molecule and an siRNA molecule.

19. The process of claim 10, wherein the nucleic acids are introduced using a transposon system.

20. The process of claim 19, wherein the transposon system is the sleeping beauty (SB) transposon system.

21. The process of claim 20, wherein the nucleic acids are introduced into the T cells via two vectors and a third vector containing a nucleic acid encoding an SB transposase is also introduced into the T cells.

22. The process of claim 16, wherein the nucleic acids are introduced using a transposon system.

23. The process of claim 22, wherein the transposon system is the sleeping beauty (SB) transposon system.

24. The process of claim 23, wherein the nucleic acids are introduced into the T cells via two vectors and a third vector containing a nucleic acid encoding an SB transposase is also introduced into the T cells.

25. The process of claim 17, wherein the nucleic acids are introduced using a transposon system.

26. The process of claim 25, wherein the transposon system is the sleeping beauty (SB) transposon system.

27. The process of claim 26, wherein the nucleic acids are introduced into the T cells via two vectors and a third vector containing a nucleic acid encoding an SB transposase is also introduced into the T cells.

28. The process of claim 18, wherein the nucleic acids are introduced using a transposon system.

29. The process of claim 28, wherein the transposon system is the sleeping beauty (SB) transposon system.

30. The process of claim 29, wherein the nucleic acids are introduced into the T cells via two vectors and a third vector containing a nucleic acid encoding an SB transposase is also introduced into the T cells.

31. A method for treating a disease associated with an antigen comprising administering a therapeutically effective amount of the genetically engineered T cells of claim 1.

32. A method for treating B-lineage acute lymphoblastic leukemia comprising administering a therapeutically effective amount of the genetically engineered T cells of claim 7.

33. A method for treating B-lineage acute lymphoblastic leukemia comprising administering a therapeutically effective amount of the genetically engineered T cells of claim 8.

34. A method for treating a disease associated with an antigen comprising administering a therapeutically effective amount of the genetically engineered T cells of claim 9.
